(19) United States
(12) Patent Application Publication
(10) Pub. No.: US 2026/0364273 A1
(43) Pub. Date: Nov. 19, 2026

(54) DEVICES AND METHODS FOR DELIVERY OF HIGH VISCOSITY FLUIDS

(71) Applicant: CoFlo Medical, Inc, San Francisco, CA (US)

(72) Inventors: Simon Rufer, Cambridge, MA (US); Vishnu Jayaprakash, Cambridge, MA (US); Kripa K. Varanasi, Lexington, MA (US)

(21) Appl. No.: 19/718,647

(22) Filed: Jun. 24, 2026

Related U.S. Application Data
(63) Continuation of application No. 19/515,677, filed on Sep. 6, 2024.
(60) Provisional application No. 63/581,610, filed on Sep. 8, 2023.

(51) Int. Cl. A61M 5/32; A61M 5/14; A61M 25/00

(52) U.S. Cl. CPC A61M 5/329; A61M 5/1407; A61M 2025/0047; A61M 2206/18

(57) ABSTRACT

An example of a device can deliver fluids having high viscosities, including fluids having viscosities of 1,000 cP or more, through a channel. The device may be or include a syringe or injector. A barrel, a hub, a needle, or a combination thereof may define at least a portion of the channel. Also disclosed herein are methods of flowing fluids having such high viscosities. In certain embodiments, a method comprises simultaneous flow of a relatively high viscosity first fluid and a relatively low lubricant through a channel, for example by using the lubricant to separate the first fluid from an interior surface of the channel, such as is the case under core annular flow. By sizing and shaping channels as disclosed herein, in particular at constrictions, stable core annular flow can be achieved, which facilitates flowing of high viscosity fluids by manual application of force at useful flow rates.

### CROSS-REFERENCE TO RELATED APPLICATIONS

This application claims priority to U.S. Provisional Patent Application No. 63/581,610, filed Sep. 8, 2023, the disclosure of which is incorporated by reference herein in its entirety.

### TECHNICAL FIELD

This disclosure relates generally to flowing high viscosity fluids through a channel. In some embodiments, the channel is comprised in a syringe and the fluid is a pharmaceutical composition.

### BACKGROUND

Biologics, including monoclonal antibodies (mAb), enzymes, peptides, and recombinant therapeutic protein formulations, have demonstrated tremendous value in treating a wide range of challenging conditions. Over the past decade, the transition from intravenous administration to subcutaneous administration has shown promise to reduce injection times from hours to minutes, lower administration costs, and improve patient experience.

The volume available in subcutaneous tissue limits maximum injection volumes to a few milliliters. In order to deliver their target doses, biologic formulations must therefore be highly concentrated and, as a result, become extremely viscous. Injection of such highly viscous drug products with conventional subcutaneous syringes and needles is impossible due to excessive force that would be required. Several methods have been proposed to enable these injections: manual injection with large needle gauges and extended time periods; multiple injections to achieve the required dosing; autoinjectors using gas or spring actuation to increase injection force; needle-free methods such as jet injectors; drug product encapsulation to reduce effective viscosity; and co-injection of hyaluronidase to increase the volume available in subcutaneous tissues.

However, these approaches have a number of disadvantages that range from poor patient experience, device complexity, and their applicability to a broad range of formulations. Slow manual injections with needles as large as 22 gauge lead to pain and patient discomfort, which compromise the patient experience. Other methods, such as jet injectors and autoinjectors, are mechanically complex, expensive, and must be customized to each drug product, which may require additional regulatory approval. In some cases, there is also potential risk of contamination with repetitive device use. Encapsulation, excipients, and hyaluronidase approaches may alter or interact with the drug product itself and introduce incompatibility risks. Costly reformulation, development, validation, and/or regulatory approval, therefore, become necessary for each individual drug product. Furthermore, encapsulation approaches may suffer from needle clogging issues and suspension stability issues, which complicate delivery and dose accuracy.

There remains a need, therefore, for methods and devices that are suitable to deliver high viscosity fluids, especially for drug delivery.

### SUMMARY

Core annular flow can be used to facilitate the transport of highly viscous drug products through needles, enabling easy injections. A relatively lower viscosity fluid can be used as a lubricant to allow a relatively higher viscosity fluid to flow through a channel. The lubricant can separate the higher viscosity fluid from an interior surface of the channel. The fluids may flow coaxially, albeit at different rates. The force required to be applied to cause the viscous fluid to flow can thus become largely agnostic to the exact viscosity of the fluid. Such channels may be, for example in a needle and hub of a syringe or injector. However, use of such a lubricant alone may only enable flowing of fluids up to certain viscosities, for example under manually applied force.

Disclosed herein, inter alia, are methods of flowing fluids of very high viscosity and devices through which fluids of different viscosities, including very high viscosities, can be flowed. For example, methods and devices disclosed herein can flow fluids having a viscosity anywhere in a range of 5 cP to 10,000 cP. Such fluid can flow under manually applied force (e.g., as applied when pinching a syringe). The present disclosure encompasses the recognition that the size and shape of a channel, in particular the size and shape of any constrictions in the channel, enables delivery of high viscosity fluids through the channel (e.g., by a syringe). Devices and channels disclosed herein can be used to flow fluids [e.g., high viscosity (e.g., at least 10 cP)] at high efficiency (η), for example greater than 20%, greater than 34%, greater than 40%, greater than 50%, greater than 52%, greater than 60%, greater than 70%, greater than 76%, greater than 80%, or greater than 90%, for example at flow rates of at least 1 mL/min. Such high efficiencies can be achieved for a channel and/or device using core annular flow, even with miscible lubricants and/or non-vertical channel and/or device orientations. Thus, the pressure requirements to achieve fluid flow using devices and/or channels disclosed herein can approach theoretical limits for laminar, ideal, fully developed, perfectly concentric core annular flow conditions, an improvement over prior devices. Thus, in some embodiments, relatively high viscosity fluids can flow under application of relatively low forces.

In some aspects, the invention is directed to a fluid delivery device comprising a channel (e.g., in a hub, optionally also a needle) extending from an inlet to an outlet. In some embodiments, the channel is sized and shaped such that a first fluid having a viscosity of at least 10 cP (e.g., at least 15 cP, greater than 20 cP, at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) can flow through the channel from the inlet (e.g., a first end of the channel and/or an initial position within the channel) to and through the outlet. In some embodiments, the channel has a curvature such that a first fluid having a viscosity of at least 10 cP (e.g., at least 15 cP, at least 20 cP, at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) can flow through the channel from the inlet (e.g., a first end of the channel and/or an initial position within the channel) to and through the outlet. In some embodiments, the channel is sized and shaped such that the first fluid can flow through the channel from the inlet (e.g., a first end of the channel and/or an initial position within the channel) to and through the outlet under a core annular flow (e.g., coaxial lubrication) (e.g., a stable core annular flow) [e.g., wherein a lubricant coats an interior surface of the channel (e.g., and is concentric and coaxial with the (more viscous) first fluid flowing through the channel), and wherein the first fluid does not contact the interior surface of the channel, e.g., wherein the lubricant has a viscosity less than 10 cP (e.g., a viscosity in a range from 0.5 cP to 10 cP, e.g., from 0.5 cP to 5 cP or 0.5 cP to 2 cP)]. In certain embodiments, a longitudinal (e.g., a major) axis of the channel lies within 45 degrees (e.g., within 10 degrees, 20 degrees, 30 degrees, or 40 degrees) of a line perpendicular to gravity. In certain embodiments, the first fluid has a viscosity of at least 10 cP (e.g., at least 15 cP, greater than 20 cP, at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP).

In some embodiments, the core annular flow has an efficiency (η) greater than 20% (e.g., greater than 34%, greater than 40%, greater than 50%, greater than 52%, greater than 60%, greater than 70%, greater than 76%, greater than 80%, or greater than 90%) (e.g., greater than 50%, greater than 52%, greater than 60%, greater than 70%, greater than 80%, or greater than 90% with an immiscible first fluid and lubricant and/or a vertical channel orientation). In some embodiments, the channel is sized and shaped such that the first fluid can flow with the efficiency when a longitudinal (e.g., major) axis of the channel lies within 45 degrees (e.g., within 10 degrees, 20 degrees, 30 degrees, or 40 degrees) of a line perpendicular to gravity. In some embodiments, the channel is sized and shaped such that the first fluid can flow with the efficiency when (e.g., only when) the first fluid and a lubricant exhibiting the core annular flow are miscible.

In certain embodiments, the channel is sized and shaped such that the first fluid can be transported through the channel by manual application of force at the inlet (e.g., with a plunger).

In certain embodiments, a force of no more than 40 N (e.g., no more than 30 N, no more than 25 N, no more than 20 N, no more than 15 N, no more than 12.5 N, or no more than 10 N) can cause the fluid to flow through the channel and through the outlet.

In certain embodiments, the channel is sized and shaped such that the first fluid having a viscosity of at least 10 cP (e.g., at least 15 cP, at least 20 cP, at least 50 cP, at least 100 cP, at least 200 cP, at least 300 cP, at least 500 cP, at least 750 cP, at least 1,000 cP, at least 1,100 cP, at least 1,200 cP, at least 1,300 cP, at least 1,500 cP, at least 2,000 cP, at least 3,000 cP, at least 4,000 cP, or at least 5,000 cP) can flow through the channel from the inlet (e.g., a first end of the channel and/or an initial position within the channel) to and through the outlet under core annular flow (e.g., coaxial lubrication) (e.g., a stable core annular flow) [e.g., wherein a lubricant coats an interior surface of the channel (e.g., and is concentric and coaxial with the (more viscous) first fluid flowing through the channel), and wherein the first fluid does not contact the interior surface of the channel, e.g., wherein the lubricant has a viscosity less than 10 cP (e.g., a viscosity in a range from 0.5 cP to 10 cP, e.g., from 0.5 cP to 5 cP or 0.5 cP to 2 cP)].

In certain embodiments, the device comprises a constriction (e.g., in a hub) in the channel disposed between the inlet and the outlet, the constriction having an interior shape that comprises a convex portion relative to an interior of the channel (e.g., wherein a second derivative of an interior profile of the constriction is positive (>0) at at least some point along the interior profile with coordinate system defined as in FIG. 3A).

In certain embodiments, the constriction has an interior shape that is entirely convex relative to the interior of the channel [e.g., having a shape defined by R(z)=(C₁z+C₂)ⁿ, where R is radius, z is position along the channel, and −1<n<−0.1 (e.g., n=−1/3)].

In certain embodiments, the constriction tapers relatively more sharply with position at a larger diameter end of the constriction and relatively less sharply with position at a smaller diameter end.

In certain embodiments, a second derivative of an interior profile of the constriction is positive (>0) at every point along the interior profile.

In certain embodiments, the channel comprises an interior surface and the device comprises the first fluid and a lubricant, each disposed in the channel, wherein the lubricant separates the first fluid from the interior surface (e.g., wherein the interior surface is textured) and the lubricant is less viscous than the first fluid.

In certain embodiments, the first fluid has a shear stress no more than 1000× (e.g., no more than 100×, no more than 10×, no more than 5×, or no more than 2×) of shear stress of the lubricant.

In certain embodiments, the first fluid and the lubricant are disposed coaxially in at least a portion of the channel.

In certain embodiments, the channel, the first fluid, and the lubricant are disposed such that, when flowing, flow of the first fluid is characterized by an eccentricity (E) with E<1. In certain embodiments, E<0.05 (e.g., E<0.02 or E=0).

In certain embodiments, the channel comprises a second inlet, (ii) the inlet is an inlet for the first fluid, and (iii) the second inlet is an inlet for the lubricant (e.g., wherein a hub that comprises at least a portion of the channel comprises the first inlet and the second inlet) (e.g., wherein the first inlet and the second inlet are spatially separated, e.g., at a hub).

In certain embodiments, a ratio of area of the inlet to area of the second inlet is in a range of from 0.02 to 5 (e.g., from 0.02 to 2, from 0.02 to 4, from 0.02 to 3, e.g., from 0.04 to 2, from 0.04 to 4, from 0.04 to 3, from 0.05 to 2, from 0.06 to 2, from 0.07 to 2, from 0.02 to 4, from 0.02 to 3, from 0.02 to 1.8, from 0.02 to 1.6, from 0.02 to 1.4, from 0.04 to 4, from 0.04 to 3, from 0.04 to 1.8, from 0.04 to 1.6, from 0.04 to 1.4, from 0.06 to 4, from 0.06 to 3, from 0.06 to 2, from 0.06 to 1.8, from 0.06 to 1.6, from 0.06 to 1.4, from 0.07 to 4, from 0.07 to 3, from 0.07 to 2, from 0.07 to 1.8, from 0.07 to 1.6, from 0.07 to 1.4, from 0.1 to 4, from 0.1 to 3, from 0.1 to 2, from 0.1 to 1.8, from 0.1 to 1.6, from 0.1 to 1.4, from 0.2 to 4, from 0.2 to 3, from 0.2 to 2, from 0.3 to 4, from 0.3 to 3, from 0.3 to 2, from 0.4 to 4, from 0.4 to 3, from 0.4 to 2, from 0.5 to 4, from 0.5 to 3, from 0.5 to 2, from 0.5 to 1.8, from 0.5 to 1.6, or from 0.5 to 1.4).

In certain embodiments, the lubricant is aqueous.

In certain embodiments, the lubricant is non-aqueous.

In certain embodiments, the lubricant comprises a buffer.

In certain embodiments, the first fluid comprises a buffer that is the same as the buffer comprised in the lubricant.

In certain embodiments, the first fluid has a viscosity in a range of from 5 cP to 10,000 cP (e.g., from 5 cP to 5,000 cP, from 5 cP to 4,000 cP, from 5 cP to 3,000 cP, from 5 cP to 2,500 cP, from 5 cP to 2,000 cP, from 20 cP to 10,000, from 20 cP to 5,000 cP, from 20 cP to 4,000 cP, from 20 cP to 3,000 cP, from 20 cP to 2,500 cP, from 20 cP to 2,000 cP, from 50 cP to 10,000, from 50 cP to 5,000 cP, from 50 cP to 4,000 cP, from 50 cP to 3,000 cP, from 50 cP to 2,500 cP, from 50 cP to 2,000 cP, from 75 cP to 10,000, from 75 cP to 5,000 cP, from 75 cP to 4,000 cP, from 75 cP to 3,000 cP, from 75 cP to 2,500 cP, from 75 cP to 2,000 cP, from 100 cP to 10,000, from 100 cP to 5,000 cP, from 100 cP to 4,000 cP, from 100 cP to 3,000 cP, from 100 cP to 2,500 cP, from 100 cP to 2,000 cP, from 250 cP to 10,000, from 250 cP to 5,000 cP, from 250 cP to 4,000 cP, from 250 cP to 3,000 cP, from 250 cP to 2,500 cP, from 250 cP to 2,000 cP, from 500 cP to 10,000, from 500 cP to 5,000 cP, from 500 cP to 4,000 cP, from 500 cP to 3,000 cP, from 500 cP to 2,500 cP, from 500 cP to 2,000 cP, from 1,000 cP to 10,000, from 1,000 cP to 5,000 cP, from 1,000 cP to 4,000 cP, from 1,000 cP to 3,000 cP, from 1,000 cP to 2,500 cP, or from 1,000 cP to 2,000 cP).

In certain embodiments, the lubricant has a viscosity in a range of from 0.5 cP to 10 cP (e.g., from 0.5 cP to 5 cP or 0.5 cP to 2 cP).

In certain embodiments, the lubricant is no more than 50 vol % (e.g., no more than 40 vol %, no more than 30 vol %, no more than 25 vol %, no more than 20 vol %, no more than 15 vol %, no more than 10 vol %, or no more than 5 vol %) of total volume of material inside the channel.

In certain embodiments, a volume ratio of the first fluid to the lubricant is no less than 1:1 (e.g., no less than 2:1, no less than 3:1, no less than 4:1, no less than 5:1, no less than 6:1, no less than 7:1, no less than 8:1, or no less than 9:1).

In certain embodiments, a volume ratio of the first fluid to the lubricant is no more than 200:1 (e.g., no more than 100:1).

In certain embodiments, the first fluid is a pharmaceutical composition (e.g., formulation) (e.g., comprising a small molecule and/or a biologic) [e.g., comprising a small molecule and/or a biologic (e.g., a monoclonal antibody (mAb), a polyclonal antibody (pAb), an enzyme, a peptide, a recombinant therapeutic protein formulation, or a combination thereof)].

In certain embodiments, the channel is sized and shaped such that fluid having viscosity anywhere in a range of at least from 5 cP to 10,000 cP (e.g., from 5 cP to 5,000 cP, from 5 cP to 4,000 cP, from 5 cP to 3,000 cP, from 5 cP to 2,500 cP, from 5 cP to 2,000 cP, from 5 cP to 1,500 cP, or from 5 cP to 1,200 cP) can flow through the channel from the inlet to and through the outlet.

In certain embodiments, the delivery device comprises a syringe [e.g., operable by manual application of force (e.g., force applied to one or more plungers)].

In certain embodiments, the syringe comprises a barrel and a hub (e.g., comprising the constriction) (e.g., a 3D printed hub) in fluid communication with the barrel (e.g., through a tube), the barrel (e.g., a tip of the barrel) and the hub each defining a portion of the channel.

In certain embodiments, the hub comprises the inlet.

In certain embodiments, the syringe comprises a needle comprising the outlet and a body of the needle defines a portion of the channel (e.g., without a substantially right angle in a fluid path through the hub and/or the needle).

In certain embodiments, a portion of the needle is disposed inside an end of the hub, the portion of the needle comprises a needle inlet, and the needle inlet comprises a gradual constriction to an inner diameter of the needle.

In certain embodiments, an outlet of the hub is disposed inside the needle.

In certain embodiments, the needle is smaller than 22 gauge (e.g., 25 gauge or 27 gauge).

In certain embodiments, no diameter of the channel is more than 5 cm (e.g., than 4 cm, than 3 cm, than 2 cm, than 1 cm, or than 0.5 cm).

In certain embodiments, the channel is sized and shaped to eliminate viscous displacement during flow of the fluid (e.g., the first fluid) through the channel.

In some aspects, the invention is directed to a method of delivering a first fluid through a channel (e.g., in a hub, optionally also a needle), the first fluid having a viscosity of at least 10 cP (e.g., at least 15 cP, greater than 20 cP, at least 50 cP, at least 100 cP, at least 200 cP, at least 300 cP, at least 500 cP, at least 750 cP, at least 1,000 cP, at least 1,100 cP, at least 1,200 cP, at least 1,300 cP, at least 1,500 cP, at least 2,000 cP, at least 3,000 cP, at least 4,000 cP, or at least 5,000 cP), the method comprising simultaneously flowing a lubricant and the first fluid through the channel and out of an outlet of the channel (e.g., into a needle or out of a needle), wherein the lubricant is less viscous than the first fluid.

In certain embodiments, the first fluid has a viscosity in a range of from 50 cP to 10,000 cP (e.g., from 50 cP to 5,000 cP, from 50 cP to 4,000 cP, from 50 cP to 3,000 cP, from 50 cP to 2,500 cP, from 50 cP to 2,000 cP, from 75 cP to 10,000, from 75 cP to 5,000 cP, from 75 cP to 4,000 cP, from 75 cP to 3,000 cP, from 75 cP to 2,500 cP, from 75 cP to 2,000 cP, from 100 cP to 10,000, from 100 cP to 5,000 cP, from 100 cP to 4,000 cP, from 100 cP to 3,000 cP, from 100 cP to 2,500 cP, from 100 cP to 2,000 cP, from 250 cP to 10,000, from 250 cP to 5,000 cP, from 250 cP to 4,000 cP, from 250 cP to 3,000 cP, from 250 cP to 2,500 cP, from 250 cP to 2,000 cP, from 500 cP to 10,000, from 500 cP to 5,000 cP, from 500 cP to 4,000 cP, from 500 cP to 3,000 cP, from 500 cP to 2,500 cP, from 500 cP to 2,000 cP, from 1,000 cP to 10,000, from 1,000 cP to 5,000 cP, from 1,000 cP to 4,000 cP, from 1,000 cP to 3,000 cP, from 1,000 cP to 2,500 cP, or from 1,000 cP to 2,000 cP).

In certain embodiments, the lubricant has a viscosity less than 10 cP (e.g., a viscosity in a range from 0.5 cP to 10 cP, e.g., from 0.5 cP to 5 cP or 0.5 cP to 2 cP).

In certain embodiments, the method comprises manually applying force to the first fluid (e.g., and the lubricant) to cause the first fluid to flow through the channel.

In certain embodiments, the force is applied at an inlet to the channel through the manual application of force at the inlet (e.g., with a plunger).

In certain embodiments, the force is no more than 40 N (e.g., no more than 30 N, no more than 25 N, no more than 20 N, no more than 15 N, no more than 12.5 N, or no more than 10 N).

In certain embodiments, the lubricant and the first fluid exhibit core annular flow (e.g., coaxial lubrication) (e.g., a stable core annular flow) while simultaneously flowing through the channel.

In some embodiments, the first fluid and the lubricant are miscible.

In certain embodiments, the core annular flow has an efficiency (η) greater than 20% (e.g., greater than 34%, greater than 40%, greater than 50%, greater than 52%, greater than 60%, greater than 70%, greater than 76%, greater than 80%, or greater than 90%) (e.g., greater than 50%, greater than 52%, greater than 60%, greater than 70%, greater than 80%, or greater than 90% with an immiscible first fluid and lubricant and/or a vertical channel orientation). In some embodiments, the first fluid flows with the efficiency when a longitudinal (e.g., major) axis of the channel lies within 45 degrees (e.g., within 10 degrees, 20 degrees, 30 degrees, or 40 degrees) of a line perpendicular to gravity. In some embodiments, the first fluid flows with the efficiency when (e.g., only when) the first fluid and the lubricant are miscible.

In certain embodiments, the method comprises flowing the lubricant and the first fluid through a constriction (e.g., hub) in the channel, the constriction having an interior shape that comprises a convex portion relative to an interior of the channel (e.g., wherein a second derivative of an interior profile of the constriction is positive (>0) at at least some point along the interior profile with coordinate system defined as in FIG. 3A).

In certain embodiments, the constriction has an interior shape that is entirely convex relative to the interior of the channel [e.g., having a shape defined by R(z)=(C₁z+C₂)ⁿ, where R is radius, z is position along the channel, and −1<n<−0.1 (e.g., n=−1/3)].

In certain embodiments, the constriction tapers relatively more sharply with position at a larger diameter end of the constriction and relatively less sharply with position at a smaller diameter end.

In certain embodiments, a second derivative of an interior profile of the constriction is positive (>0) at every point along the interior profile.

In certain embodiments, a shear stress of the first fluid is no more than 1000× (e.g., no more than 100×, no more than 10×, no more than 5×, or no more than 2×) of shear stress of the lubricant during the flow.

In certain embodiments, the lubricant separates the first fluid from an interior surface of the channel (e.g., wherein the first fluid and the lubricant are disposed coaxially in at least a portion of the channel).

In certain embodiments, flow of the first fluid is characterized by an eccentricity (E) with E<1.

In certain embodiments, E<0.05 (e.g., E<0.02 or E=0).

In certain embodiments, the method comprises providing (e.g., simultaneously) the first fluid through a first inlet to the channel and the lubricant through a second inlet to the channel.

In certain embodiments, the lubricant is aqueous. In certain embodiments, the lubricant is non-aqueous. In certain embodiments, the lubricant comprises a buffer. In certain embodiments, the first fluid comprises a buffer that is the same as the buffer comprised in the lubricant.

In certain embodiments, the lubricant is no more than 50 vol % (e.g., no more than 40 vol %, no more than 30 vol %, no more than 25 vol %, no more than 20 vol %, no more than 15 vol %, no more than 10 vol %, or no more than 5 vol %) of total volume of material inside the channel.

In certain embodiments, a volume ratio of the first fluid to the lubricant is no less than 1:1 (e.g., no less than 2:1, no less than 3:1, no less than 4:1, no less than 5:1, no less than 6:1, no less than 7:1, no less than 8:1, or no less than 9:1).

In certain embodiments, a volume ratio of the first fluid to the lubricant is no more than 200:1 (e.g., no more than 100:1).

In certain embodiments, the first fluid is a pharmaceutical composition (e.g., formulation) [e.g., comprising a small molecule and/or a biologic (e.g., a monoclonal antibody (mAb), a polyclonal antibody (pAb), an enzyme, a peptide, a recombinant therapeutic protein formulation, or a combination thereof)] (e.g., and the method comprises subcutaneously administering the pharmaceutical composition through the channel).

In certain embodiments, a syringe [e.g., operable by manual application of force (e.g., to one or more plungers)] comprises the channel.

In certain embodiments, the syringe comprises a barrel and a hub (e.g., comprising the constriction) (e.g., a 3D printed hub) in fluid communication with the barrel (e.g., through a tube), the barrel (e.g., a tip of the barrel) and the hub each defining a portion of the channel.

In certain embodiments, the barrel comprises an inlet for the first fluid (e.g., and a separate inlet for the lubricant).

In certain embodiments, the syringe comprises a needle comprising the outlet and a body of the needle defines a portion of the channel.

In certain embodiments, a portion of the needle is disposed inside an end of the hub, the portion of the needle comprises a needle inlet, and the needle inlet comprises a gradual constriction to an inner diameter of the needle.

In certain embodiments, an outlet of the hub is disposed inside the needle.

In certain embodiments, the needle is smaller than 22 gauge (e.g., 25 gauge or 27 gauge).

In certain embodiments, no diameter of the channel is more than 5 cm (e.g., than 4 cm, than 3 cm, than 2 cm, than 1 cm, or than 0.5 cm).

In certain embodiments, the first fluid is flowed through the channel such that viscous displacement does not occur.

In certain embodiments, flow of the first fluid occurs at a rate at least 2× (e.g., at least 3×, or at least 4×) of a rate of flow of the lubricant.

In certain embodiments, the flow of the first fluid occurs at a rate no more than 5× of a rate of the flow of the lubricant.

In certain embodiments, flow of the first fluid occurs at a rate of at least 1 mL/min (e.g., at least 2 mL/min, at least 3 mL/min, at least 4 mL/min, at least 5 mL/min, at least 6 mL/min, at least 7 mL/min, at least 8 mL/min, at least 9 mL/min, or at least 10 mL/min).

In certain embodiments, the flow of the first fluid occurs at a rate of no more than 20 mL/min.

In certain embodiments, flow of the lubricant occurs at a rate of at least 0.5 mL/min (e.g., at least 1 mL/min, at least 1.5 mL/min, at least 2 mL/min, or at least 2.5 mL/min).

In certain embodiments, the flow of the lubricant occurs at a rate of no more than 3 mL/min.

In some aspects, the invention is directed to a fluid delivery hub (e.g., plastic hub) (e.g., 3D printed hub) comprising a channel extending from an inlet of the hub to an outlet of the hub, the channel comprising a constriction, the constriction having an interior shape that comprises a convex portion relative to an interior of the channel (e.g., wherein a second derivative of an interior profile of the constriction is positive (>0) at at least some point along the interior profile with coordinate system defined as in FIG. 3A).

In certain embodiments, the channel is sized and shaped such that a first fluid having a viscosity greater than 10 cP (e.g., at least 15 cP, at least 20 cP, at least 50 cP, at least 100 cP, at least 200 cP, at least 300 cP, at least 500 cP, at least 750 cP, at least 1,000 cP, at least 1,100 cP, at least 1,200 cP, at least 1,300 cP, at least 1,500 cP, at least 2,000 cP, at least 3,000 cP, at least 4,000 cP, or at least 5,000 cP) can flow through the channel from the inlet (e.g., a first end of the channel and/or an initial position within the channel) to and through the outlet.

In certain embodiments, the channel is sized and shaped such that fluid having viscosity anywhere in a range of at least from 5 cP to 10,000 cP can flow through the channel from the inlet to and through the outlet.

In certain embodiments, the constriction has an interior shape that is entirely convex relative to the interior of the channel [e.g., having a shape defined by R(z)=(C₁z+C₂)ⁿ, where R is radius, z is position along the channel, and −1<n<−0.1 (e.g., n=−1/3)].

In certain embodiments, the constriction tapers relatively more sharply with position at a larger diameter end of the constriction and relatively less sharply with position at a smaller diameter end.

In certain embodiments, a second derivative of a profile of the constriction is positive (>0) at every point along the profile.

In some aspects, the invention is directed to an apparatus comprising the hub and a needle attached to the hub, wherein a lumen of the needle is in fluid communication with the channel.

In certain embodiments, an inlet to the needle is disposed inside of the channel.

In certain embodiments, the needle inlet comprises a gradual constriction to an inner diameter of the lumen.

In certain embodiments, the outlet of the hub is disposed inside the lumen.

In certain embodiments, the needle is smaller than 22 gauge (e.g., 25 gauge or 27 gauge).

In some aspects, the invention is directed to a fluid delivery device comprising a channel extending from an inlet to an outlet, wherein the channel is sized and shaped such that fluid having viscosity anywhere in a range of at least from 5 cP to 10,000 cP (e.g., from 5 cP to 5,000 cP, from 5 cP to 4,000 cP, from 5 cP to 3,000 cP, from 5 cP to 2,500 cP, from 5 cP to 10,000 cP, from 10 cP to 5,000 cP, from 10 cP to 4,000 cP, from 10 cP to 3,000 cP, from 10 cP to 2,500 cP, from 25 cP to 5,000 cP, from 25 cP to 4,000 cP, from 25 cP to 3,000 cP, from 25 cP to 2,500 cP, from 25 cP to 2,000 cP, from 50 cP to 10,000 cP, from 50 cP to 5,000 cP, from 50 cP to 4,000 cP, from 50 cP to 3,000 cP, from 50 cP to 2,500 cP, from 50 cP to 2,000 cP, from 75 cP to 10,000, from 75 cP to 5,000 cP, from 75 cP to 4,000 cP, from 75 cP to 3,000 cP, from 75 cP to 2,500 cP, from 75 cP to 2,000 cP, from 100 cP to 10,000, from 100 cP to 5,000 cP, from 100 cP to 4,000 cP, from 100 cP to 3,000 cP, from 100 cP to 2,500 cP, from 100 cP to 2,000 cP, from 250 cP to 10,000, from 250 cP to 5,000 cP, from 250 cP to 4,000 cP, from 250 cP to 3,000 cP, from 250 cP to 2,500 cP, from 250 cP to 2,000 cP, from 500 cP to 10,000, from 500 cP to 5,000 cP, from 500 cP to 4,000 cP, from 500 cP to 3,000 cP, from 500 cP to 2,500 cP, from 500 cP to 2,000 cP, from 1,000 cP to 10,000, from 1,000 cP to 5,000 cP, from 1,000 cP to 4,000 cP, from 1,000 cP to 3,000 cP, from 1,000 cP to 2,500 cP, or from 1,000 cP to 2,000 cP) can flow through the channel from the inlet (e.g., a first end of the channel and/or an initial position within the channel) to and through the outlet.

In some aspects, the invention is directed to a device (e.g., a syringe comprising a barrel, a hub, and optionally a needle) for delivery of a fluid, wherein the device is shaped to establish a core annular flow (e.g., coaxial lubrication) for delivery of the fluid (e.g., a fluid with a viscosity of at least 5 cP, e.g., at least 10 cP, e.g., at least 20 cP, e.g., at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) from a barrel of the device (e.g., and through a hub and/or needle of the device) through an outlet (e.g., via operation of a plunger, e.g., via mechanical operation of the plunger by a user, e.g., by manual force) (e.g., delivery of the fluid to a subject) when the fluid is flowed through the device.

In certain embodiments, flow of the fluid is characterized by an eccentricity (E) with E<1 (e.g., E<0.05, e.g., E<0.02, e.g., E=0).

In certain embodiments, the fluid is a viscous drug formulation and wherein the device is shaped to establish stable core annular flow for delivery of the fluid to a subject when the fluid is flowed through the device.

In certain embodiments, flow of the fluid is characterized by an eccentricity (E) with E<1 (e.g., E<0.05, e.g., E<0.02, e.g., E=0).

In some aspects, the invention is directed to a device (e.g., a syringe comprising a barrel, a hub, and optionally a needle) for delivery of a viscous drug formulation, wherein the device is shaped to establish a core annular flow (e.g., coaxial lubrication) that enables delivery of the viscous drug formulation (e.g., a drug formulation with a viscosity of at least 5 cP, e.g., at least 10 cP, e.g., at least 20 cP, e.g., at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) from a barrel of the device (e.g., and through a hub and/or needle of the device) through an outlet (e.g., via operation of a plunger, e.g., via mechanical operation of the plunger by a user, e.g., by hand) (e.g., delivery of the formulation to a subject) when the fluid is flowed through the device, wherein a shear stress of an inner fluid comprising (e.g., consisting essentially of, e.g., consisting of) the viscous drug formulation is not significantly greater than (e.g., not greater by a factor of 1000, not greater by a factor of 100, not greater by a factor of 10, not greater by a factor of 5, not greater by a factor of 3, not greater by a factor of 2, not greater by a factor of 1.1, or not greater by a factor of 1.0 than) a shear stress of an outer lubricating fluid.

In certain embodiments, the device comprises a hub with a convex profile (e.g., as opposed to a concave profile).

In some aspects, the invention is directed to a device (e.g., a syringe comprising a barrel, a hub, and optionally a needle) for delivery of a fluid, wherein the device is shaped to establish a core annular flow for delivery of the fluid (e.g., a fluid with a viscosity of at least 5 cP, e.g., at least 10 cP, e.g., at least 20 cP, e.g., at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) from a barrel of the device (e.g., and through a hub and/or needle of the device) through an outlet (e.g., via operation of a plunger, e.g., via mechanical operation of the plunger by a user, e.g., by hand) (e.g., delivery of the fluid to a subject) when the fluid is flowed through the device, wherein the core annular flow is not fully eccentric (e.g., flow of the fluid is characterized by an eccentricity (E) with E<1, e.g., E<0.05, e.g., E<0.02, e.g., E=0), and wherein throughput flow (e.g., flow through the outlet) is greater than (e.g., is at least twice, e.g., is at least 5 times, e.g., is at least 10 times) total compliance flow (e.g., compliance flow throughout all sections of the core annular flow).

In some aspects, the invention is directed to a device for delivery of a fluid (e.g., the device of any of the embodiments described herein), wherein the device is shaped to establish core annular flow that is not fully eccentric and has relevant resistance and compliance components designed to stabilize the core annular flow (e.g., dynamic system of core annular flow) when the fluid is flowed through the device.

In some aspects, the invention is directed to a device for delivery of a fluid (e.g., the device of any one of the embodiments described herein), wherein the device is shaped to establish core annular flow that is not fully eccentric and matches a resistance of lubricant fluidic pathway(s) to a resistance of viscous fluidic pathway(s) to within 1000% (e.g., wherein a ratio of the greater to the lesser of the two resistances is 10 or less, e.g., is 5 or less, e.g., is 3 or less, e.g., is 2 or less, e.g., is 1.5 or less, e.g., is 1.2 or less, e.g., is 1.1 or less) or to within 10000% (e.g., wherein a ratio of the greater to the lesser of the two resistances is 100 or less, e.g., is 50 or less, e.g., is 30 or less, e.g., is 20 or less, e.g., is 10 or less, e.g., is 5 or less, e.g., is 2 or less) when the fluid is flowed through the device.

In some aspects, the invention is directed to a device for delivery of a fluid (e.g., the device of any one of the embodiments described herein), wherein the device is shaped to establish core annular flow that is not fully eccentric and wherein the device comprises one or more fluidic dampers to isolate compliant elements, thereby reducing (e.g., preventing) viscous displacement due to compliance flow, when the fluid is flowed through the device.

In some aspects, the invention is directed to a device for delivery of a fluid (e.g., the device of any one of the embodiments described herein), wherein the device is shaped to establish core annular flow that is not fully eccentric and wherein the device matches an upstream pressure of a lubricant reservoir and a viscous fluid reservoir when the fluid is flowed through the device.

In some aspects, the invention is directed to a device (e.g., a syringe comprising a barrel, a hub, and optionally a needle) (e.g., the device of any one of the embodiments described herein), wherein the device is shaped to establish a core annular flow (e.g., coaxial lubrication) for delivery of the viscous fluid (e.g., a fluid with a viscosity of at least 5 cP, e.g., at least 10 cP, e.g., at least 20 cP, e.g., at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) from a barrel of the device (e.g., and through a hub and/or needle of the device) through an outlet (e.g., via operation of a plunger, e.g., via mechanical operation of the plunger by a user, e.g., by hand) (e.g., delivery of the fluid to a subject) when the fluid is flowed through the device, and wherein the device comprises one or more features designed to perform one or more of (i) to (iv) as follows: (i) reduce and/or minimize lubricant compliance (Cₗ); (ii) correlate (e.g., match to within a factor of no greater than 10,000, no greater than 3000, no greater than 1000, no greater than 100, no greater than 10, no greater than 5, no greater than 3, no greater than 2, no greater than 1.5, no greater than 1.2, or no greater than 1.1) the upstream resistances of the lubricant and viscous fluid (Rₗ and Rᵥ); (iii) reduce and/or minimize pressure disturbances (∂P/∂t); and (iv) reduce and/or minimize core annular flow resistance (e.g., maximize throughput flow and/or minimize compliance flow ∂v/∂t).

In certain embodiments, the device comprises one or more features designed to reduce and/or minimize lubricant compliance (C₁) defined as

$$C l = \frac{\partial V}{\partial P}$$

(change in volume per change in pressure), wherein C₁ is no greater than

$$1 {0}^{- 11} [ \frac{{s}^{2} \cdot {m}^{4}}{k g} ]$$

(e.g., no greater than 3×10⁻¹², no greater than 1×10⁻¹², no greater than 3×10⁻¹³, no greater than 5×10⁻¹³, or no greater than

$$1 \times {10}^{- 14} [ \frac{{s}^{2} \cdot {m}^{4}}{k g} ] ) .$$

In certain embodiments, the one or more features designed to reduce or minimize lubricant compliance comprises a mechanism for elimination of air bubbles in the device (e.g., an aerophobic surface and/or thin fluid passageways that are less likely to entrap air bubbles).

In certain embodiments, the device comprises one or more features designed to correlate (e.g., match to within a factor of no greater than 10, no greater than 5, no greater than 3, no greater than 2, no greater than 1.5, no greater than 1.2, or no greater than 1.1) the upstream resistances of the lubricant and viscous fluid (Rₗ and Rᵥ).

In certain embodiments, the device comprises a viscous fluid passageway and a lubricant passageway, wherein the one or more features comprises a choke point within the lubricant passageway (e.g., a resistive region in the form of a thin tube and/or a filter, e.g., comprising densely packed elements with small pores) (e.g., wherein the lubricant passageway has, within a particular region, a substantially smaller cross-sectional area than the viscous passageway). (e.g., see FIG. 10A, e.g., wherein R is defined as follows: ΔPupstream, l=Rₗ·Qₗ and ΔPupstream, v=Rᵥ·Qᵥ, where ΔPupstream refers to the pressure drop between the fluid reservoir and the initiation of core annular flow. Rᵥ and Rₗ should be correlated given the relative flow rates of viscous fluid and lubricant such that ΔPupstream, v and ΔPupstream,l are not too different, e.g., wherein ΔPupstream, v/ΔPupstream,l is no greater than about 100,000, e.g., no greater than about 10,000, e.g., no greater than about 1000, e.g., no greater than 100, e.g., no greater than about 50, e.g., no greater than about 20, e.g., no greater than 10, e.g., no greater than 5) (e.g., wherein Rᵥ/Rₗ is about 1 within an order of magnitude, e.g., within a factor of 5, e.g., within a factor of 2, e.g., within a factor of 1.5, e.g., within a factor of 1.1).

In certain embodiments, the device comprises one or more features designed to reduce and/or minimize pressure disturbances (∂P/∂t).

In certain embodiments, the device comprises a lubricant reservoir and a viscous reservoir, and wherein the one or more features designed to reduce and/or minimize disturbances comprises a common (e.g., single) pressure headspace that is shared between the lubricant and viscous reservoirs.

In certain embodiments, the one or more features designed to reduce and/or minimize disturbances comprises a passageway or other region with diameter sufficiently small to incur viscous losses (e.g., a passageway with diameter no greater than 2 mm, no greater than 1 mm, no greater than 0.5 mm, no greater than 0.2 mm, or about 0.2 mm).

In certain embodiments, the device comprises a hub that initiates the core annular flow, wherein the one or more features designed to reduce and/or minimize disturbances comprises a damping element placed between the hub and a compliant upstream element (e.g., the most compliant upstream element) (e.g., thereby limiting pressure disturbances experienced by the compliant upstream element, reducing likelihood of viscous displacement).

In certain embodiments, the device comprises one or more features designed to reduce and/or minimize core annular flow resistance (e.g., maximize throughput flow and/or minimize compliance flow ∂V/∂t).

In certain embodiments, the one or more features designed to reduce and/or minimize core annular flow resistance comprises a convex hub and/or a smooth profile (e.g., a profile with minimal or no discontinuities, e.g., a profile with a continuous (e.g., substantially continuous) derivative).

In some embodiments, the core annular flow has an efficiency (η) greater than 20% (e.g., greater than 34%, greater than 40%, greater than 50%, greater than 52%, greater than 60%, greater than 70%, greater than 76%, greater than 80%, or greater than 90%) (e.g., greater than 50%, greater than 52%, greater than 60%, greater than 70%, greater than 80%, or greater than 90% with an immiscible first fluid and lubricant and/or a vertical channel orientation). In some embodiments, the channel is sized and shaped such that the first fluid can flow with the efficiency when a longitudinal (e.g., major) axis of the channel lies within 45 degrees (e.g., within 10 degrees, 20 degrees, 30 degrees, or 40 degrees) of a line perpendicular to gravity. In some embodiments, the channel is sized and shaped such that the first fluid can flow with the efficiency when (e.g., only when) the first fluid and a lubricant exhibiting the core annular flow are miscible.

Any two or more of the features described in this specification, including in this summary section, may be combined to form implementations of the disclosure, whether specifically expressly described as a separate combination in this specification or not.

### BRIEF DESCRIPTION OF THE DRAWINGS

The present teachings described herein will be more fully understood from the following description of various illustrative embodiments, when read together with the accompanying drawings. It should be understood that the drawing described below is for illustration purposes only and is not intended to limit the scope of the present teachings in any way. The foregoing and other objects, aspects, features, and advantages of the disclosure will become more apparent and may be better understood by referring to the following description taken in conjunction with the accompanying drawings, in which:

FIG. 1A is an illustration of a syringe (with a hub and needle assembly separated from a barrel for illustration purposes), according to illustrative embodiments of the present disclosure;

FIG. 1B is a flow diagram of a method of delivering fluid, according to illustrative embodiments of the present disclosure;

FIG. 2 is a scheme illustrating core annular flow (panel A), a comparison of injection force (as a function of viscosity) needed for a conventional syringe as compared to a syringe as disclosed herein (panel B), a plot of experimentally derived injection force over time for a 500 cP first fluid (panel C) and for a 1266 cP first fluid (panel D), according to illustrative embodiments of the present disclosure;

FIG. 3 illustrates a schematic of a hub (panel A) with labelled dimensions and a photograph of a prototype hub with a construction (panel B), according to illustrative embodiments of the present disclosure;

FIG. 4 is a schematic illustrating concentric annular flow, fully eccentric annular flow, and partially eccentric annular flow, according to illustrative embodiments of the present disclosure;

FIG. 5 illustrates viscous displacement and core annular flow in photographs and in plots of pressure gradients over time, according to illustrative embodiments of the present disclosure;

FIG. 6 illustrates a prototype hub, according to illustrative embodiments of the present disclosure;

FIG. 7 illustrates a simplify diagram of an injector which uses lubricant to ease transport of a viscous fluid using core annular flow with compliances, resistances, and pressures indicated, according to illustrate embodiments of the present disclosure;

FIG. 8 illustrates a core annular flow device which has individual viscous and lubricant reservoirs and a hub where core annular flow is established, with relevant compliances being illustrated left and right of the device (left) and expressions for estimating the magnitude of physical compliance (right), according to illustrate embodiments of the present disclosure;

FIG. 9 illustrates a qualitative performance comparison of a low compliance tube and high compliance tube for a core annular flow hub, according to illustrate embodiments of the present disclosure;

FIG. 10A illustrates ways of matching resistance for simultaneous flow of a viscous fluid and a lubricant, according to illustrate embodiments of the present disclosure;

FIG. 10B illustrates a comparison of upstream versus downstream compliance in a device with two reservoirs, according to illustrative embodiments of the present disclosure;

FIG. 11 illustrates simulation results for constriction profiles (e.g., in a hub), according to illustrative embodiments of the present disclosure;

FIG. 12 illustrates additional simulation results for static pressure (panel A) and pressure gradient (panel B) for a hub profile with n=−1/3 and for static pressure (panel C) and pressure gradient (panel D) for a hub profile with n=3, according to illustrative embodiments of the present disclosure;

FIG. 13 compares a conventional needle-hub interface (panel A) to alternative needle-hub interfaces (panels B-C) that mitigate or eliminate issues caused by sudden constriction present in conventional needle-hub interfaces, according to illustrative embodiments of the present disclosure; and

FIG. 14 illustrates photographs of exemplary hubs having different profiles, according to illustrative embodiments of the present disclosure (panels A-C) and a plot of Qₒ/Qᵢ before viscous displacement vs. first fluid viscosity for the three exemplary hubs (panel D), according to illustrative embodiments of the present disclosure.

FIG. 15 illustrates the ideal pressure reduction factor for various values of Qₒ/Qᵢ, under laminar fully developed conditions, according to illustrative embodiments of the present disclosure.

FIG. 16 illustrates pressure values in a core annular flow device under various conditions, according to illustrative embodiments of the present disclosure.

Figures are not necessarily drawn to scale.

### DETAILED DESCRIPTION OF CERTAIN EMBODIMENTS

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems according to certain embodiments of the present disclosure that consist essentially of, or consist of, the recited components, and that there are processes and methods according to certain embodiments of the present disclosure that consist essentially of, or consist of, the recited processing steps.

It should be understood that the order of steps or order for performing certain action is immaterial so long as operability is not lost. Moreover, two or more steps or actions may be conducted simultaneously. As is understood by those skilled in the art, the terms “over”, “under”, “above”, “below”, “beneath”, and “on” are relative terms and can be interchanged in reference to different orientations of the layers, elements, and substrates included in the present disclosure. For example, a first layer on a second layer, in some embodiments means a first layer directly on and in contact with a second layer. In other embodiments, a first layer on a second layer can include another layer there between.

In this application, unless otherwise clear from context or otherwise explicitly stated, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; (iv) the terms “about” and “approximately” may be understood to permit standard variation as would be understood by those of ordinary skill in the relevant art; and (v) where ranges are provided, endpoints are included. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Disclosed herein are, inter alia, devices and components of such devices that enable delivery of fluids having high viscosities, including fluids having viscosities of 1,000 cP or more, through a channel. The channel may be in a device, such as a syringe or injector. A barrel, a hub, a needle, or a combination thereof may define at least a portion of the channel. Also disclosed herein are methods of flowing fluids having high viscosities, including fluids having viscosities of 1,000 cP or more, through a channel. In certain embodiments, a method comprises simultaneous flow of a relatively high viscosity first fluid (e.g., comprising a pharmaceutical composition) and a relatively low lubricant through a channel, for example by using the lubricant to separate the first fluid from an interior surface of the channel, such as is the case under core annular flow (e.g., coaxial lubrication). Relevant discussion of core annular flow (e.g., coaxial lubrication) can be found in Jayaprakash, V. et al., Enhancing the Injectability of High Concentration Drug Formulations Using Core Annular Flows, Adv. Healthcare Mater., 9 (18): 2001022 (2020), https://doi.org/10.1002/adhm.202001022, the disclosure of which is hereby incorporated by reference herein in its entirety. By sizing and shaping channels as disclosed herein, in particular at constrictions (e.g., in a hub that narrows a channel from a size of a barrel tip down to a size of a needle), stable core annular flow can be achieved, which facilitates flowing of high viscosity fluids (e.g., pharmaceutical compositions) by manual application of force at useful flow rates (e.g., more than 1 mL/min). By sizing and shaping channels as disclosed herein, in particular having a curvature (e.g., in a hub that narrows a channel from a size of a barrel tip down to a size of a needle), stable core annular flow can be achieved, which facilitates flowing of high viscosity fluids (e.g., pharmaceutical compositions) by manual application of force at useful flow rates (e.g., more than 1 mL/min).

In some embodiments, a fluid delivery device comprises a channel (e.g., in a hub, optionally also a needle) extending from an inlet to an outlet. In some embodiments, the channel is sized and shaped such that a first fluid having a viscosity of at least 5 cP (e.g., at least 10 cP) can flow through the channel from the inlet (e.g., a first end of the channel and/or an initial position within the channel) to and through the outlet, for example under core annular flow. In some embodiments, the channel is sized and shaped such that the first fluid having a viscosity greater than 20 cP can flow through the channel from the inlet to and through the outlet. In some embodiments, the channel is sized and shaped such that the first fluid having a viscosity at least 15 cP can flow through the channel from the inlet to and through the outlet. In some embodiments, the channel has a curvature such that a first fluid having a viscosity of at least 5 cP, (e.g., at least 10 cP, at least 15 cP, greater than 20 cP, at least 50 cP, at least 100 cP, at least 200 cP, at least 300 cP, at least 500 cP, at least 750 cP, at least 1,000 cP, at least 1,100 cp, at least 1,200 cP, at least 1,300 cP, at least 1,500 cP, at least 2,000 cP, at least 3,000 cP, at least 4,000 cP, or at least 5,000 cP) can flow through the channel from the inlet (e.g., a first end of the channel and/or an initial position within the channel) to and through the outlet, for example under core annular flow.

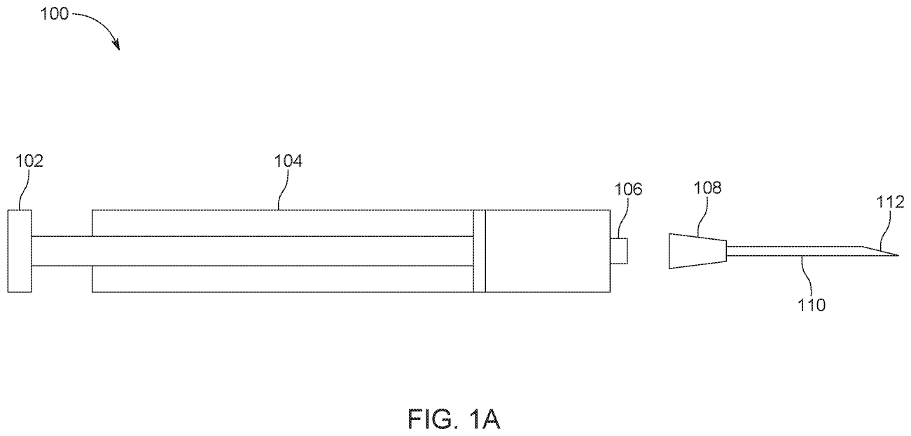

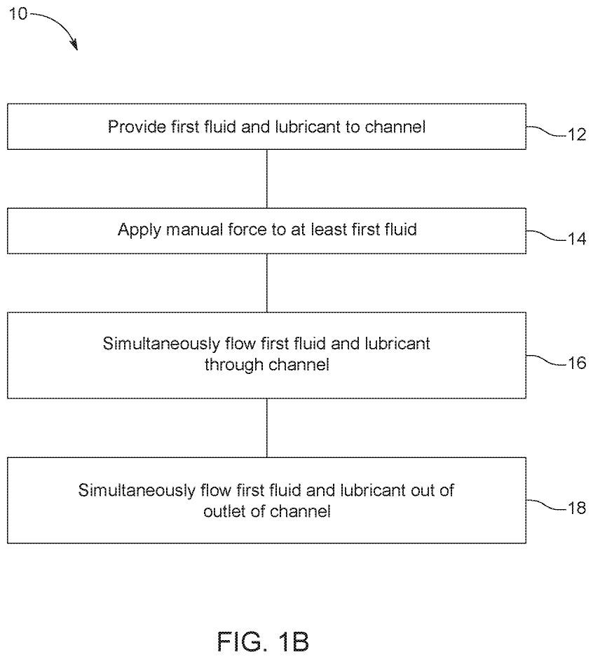

FIG. 1A illustrates an exemplary syringe 100. Syringe 100 includes a plunger 102, barrel 104, hub 108, and needle 110. Barrel 104 includes a tip 106 with an outlet. Hub 108 includes an inlet and an outlet to needle 110. Needle 110 includes a body with a lumen running therethrough to an outlet 112. Needle 110 is attached to hub 108. Hub 108 can be removably attached to tip 106 but is shown as separated for illustration purposes. Barrel 104 includes a channel. Hub 108 includes a channel. Needle 110 includes a channel in fluid communication with hub 18. Barrel 104 is in fluid communication with hub 108 and in fluid communication with needle 110 through hub 108. Hub 108 is in fluid communication with needle 110. Syringe 100 can be said to comprise a channel that is defined in part by one or more of barrel 104, hub 108, and needle 110. In some embodiments, a channel is defined solely by a hub and a needle (e.g., hub 108 and needle 110). In some embodiments, a channel is defined solely by a hub (e.g., hub 108). Hub 108 includes a constriction that has a largest diameter corresponding to a size of tip 106 and a smallest diameter corresponding to a size of the lumen of needle 110. Hub 108 may be 3D printed. Barrel 104 may include a constriction. Needle 110 may include a constriction. A first fluid and a lubricant can be provided to hub 108 and subsequently delivered through needle 110 and out of outlet 112 using barrel 104. In some embodiments, the first fluid and the lubricant flow under core annular flow. In some embodiments, a hub comprises an inlet for a channel. In some embodiments, a syringe or injector comprises a needle comprising an outlet for a channel and a body of the needle defines a portion of the channel (e.g., without a substantially right angle in a fluid path through the hub and/or the needle).

Lubricant may wet an interior surface of a channel of a device (e.g., of a hub). An interior surface of a channel may be textured to promote wetting by low viscosity fluid (e.g., lubricant).

In some embodiments, a double barrel syringe is used to deliver fluid. For example, an inner barrel may be used to deliver a first fluid (e.g., comprising a pharmaceutical composition) and an outer barrel may be used to deliver a lubricant. Core annular flow of such fluid may begin at or before an outlet of a common tip of the double barrel or in a hub to which the double barrel is connected.

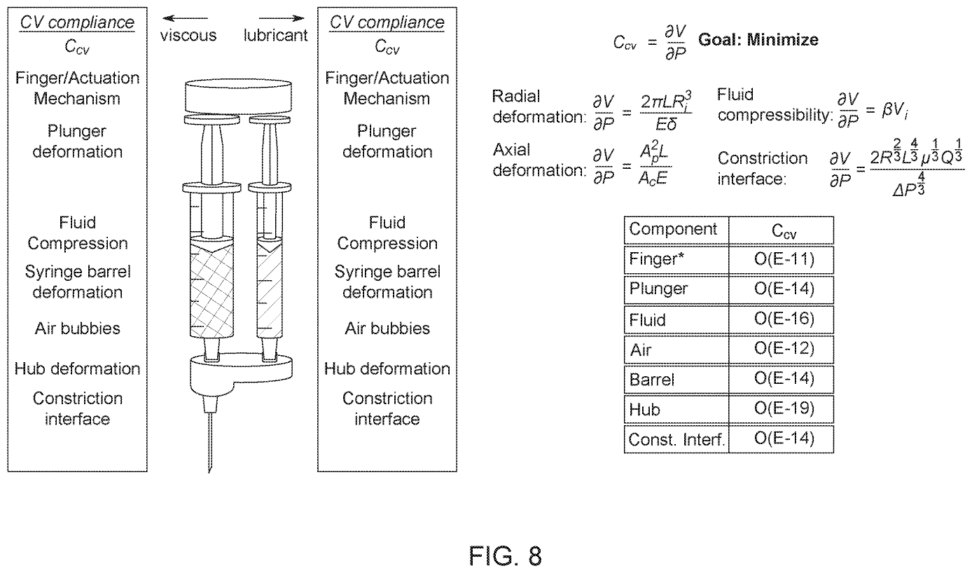

In some embodiments, a device (e.g., a syringe) includes a hub and two fluid reservoirs, one for lubricant and one for viscous fluid. The hub may include two inlets, each in fluid communication with one of the fluid reservoirs. The inlets may be spatially separated (e.g., are not concentric) on the hub (e.g., across a surface of the hub). Thus, fluid from each fluid reservoir may flow simultaneously into the hub, for example using pressure applied by plungers to the fluid reservoirs. Core annular flow may initiate in the hub. The hub may define at least a portion of a channel. The hub may include as well as a constriction to induce core annular flow. In some embodiments, pressure can be applied to both reservoirs simultaneously, and optionally substantially equally, (as shown) in order for viscous fluid and lubricant to flow into and through the hub. A hub may be in fluid communication with a needle, for example on an opposite side of the hub from the fluid reservoirs. Fluid flowed from the fluid reservoirs into the hub may, if a needle is present, then flow into and out of a needle. Core annular flow may be established in the hub during fluid flow, due at least in part to a constriction in the hub, and persist through a needle, if present. The hub may be enclosed, for example include an enclosed body that includes two inlets (one for each fluid reservoir) and one outlet (e.g., in fluid communication with a needle). FIG. 8 illustrates an example of a two reservoir plus hub syringe device that also includes a needle.

A syringe or injector may be operable by manual application of force (e.g., force applied to one or more plungers of a syringe). In some embodiments, a force of no more than 40 N (e.g., no more than 30 N, no more than 25 N, no more than 20 N, no more than 15 N, no more than 12.5 N, or no more than 10 N) can cause the fluid to flow through a channel and through (e.g., out of) an outlet of the channel (e.g., an outlet of a needle). Manual force may be applied to a first fluid to cause it to flow through a channel in any suitable fashion, including, in some embodiments where a device comprising the channel is a syringe, by pushing a component of the device (e.g., a plunger) with a hand (e.g., using a pinching motion, for example with a plunger and one or more tabs disposed on an end of a barrel).

FIG. 1B illustrates example method 10 of delivering a first fluid through a channel. In step 12, a first fluid and a lubricant are provided to the channel (e.g., via one or two inlets). The channel may be in a hub or a hub may define at least a portion of the channel (e.g., optionally in combination with a needle). In step 14, manual force is applied to at least the first fluid. This may be accomplished by, for example, pushing a plunger (e.g., with a pinching motion) that contacts the first fluid in a case where the channel is comprised in a syringe. In some embodiments, manual force is also applied (e.g., simultaneously) to the lubricant. In step 16, as a result of the manually applied to at least the first fluid, the first fluid and the lubricant are simultaneously flowed through the channel. In step 18, the first fluid and the lubricant are simultaneously flowed to and through an outlet of the channel, such as an outlet of a needle, thereby delivering the first fluid. Steps 16 and 18 may occur simultaneously. Where a syringe is used, the outlet may be an outlet of a needle, and the resulting delivery of method 10 may be a transdermal and/or subcutaneous delivery of the first fluid. Steps 16 and 18 may occur under core annular flow (e.g., coaxial lubrication). Step 16 may include simultaneously flowing the first fluid and the lubricant through a constriction in the channel (e.g., in a hub, such as a hub for a needle in a syringe).

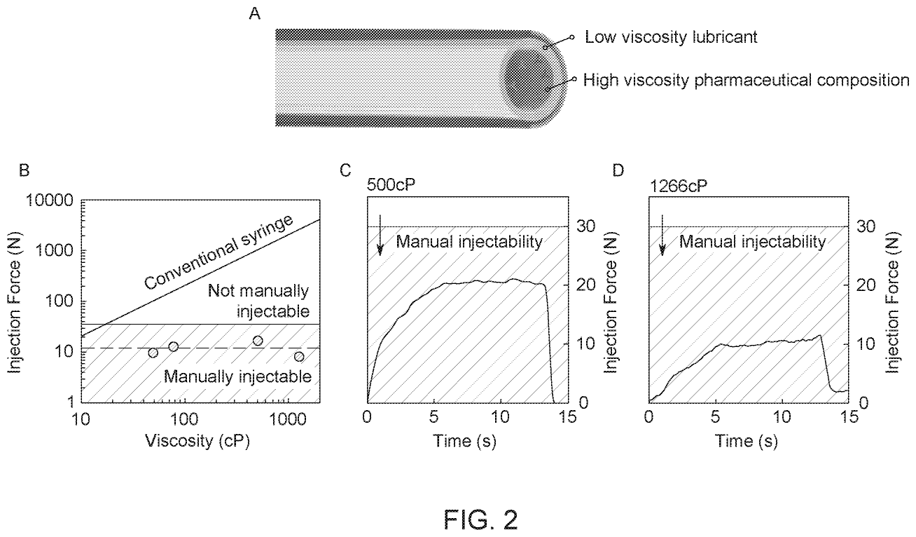

As discussed throughout the subsequent paragraphs, due to several flow instabilities, establishing a stable co-axial flow in a channel is non-trivial. A stable co-axial lubrication state can be achieved by tuning important flow parameters, and injection forces (e.g., for a syringe) can be reduced by as much as several orders of magnitude (e.g., down to manually achievable levels). The described devices (e.g., syringes and injectors) can maintain stable coaxial lubrication and unlock extremely low injection forces for highly viscous formulations: solutions as viscous as 1200 cP can be easily and rapidly injected manually, through a ½″ 27-gauge needle FIG. 2 panels B-C with injection forces significantly below 30 N.

Any low-viscosity aqueous or non-aqueous formulation can be used as a lubricant. A first fluid's buffer solution itself (if present) can be used as the lubricating liquid to ensure that delivered fluid (e.g., injected product) remains qualitatively unchanged, thereby eliminating the need for reformulation. The fraction of total delivered (e.g., injected) volume (e.g., comprising buffer solution) can be held to as low as 10%, for example to maximize total dose of a pharmaceutical composition included in the first fluid delivered.

The use of a first fluid's buffer as a lubricant to establish core annular flow may slightly dilute final concentration of delivered first fluid (e.g., pharmaceutical composition). Use of core annular flow may yield significantly greater force reductions (e.g., >5×, >10×, >15×, or >20×) compared to a diluted first fluid. Devices (e.g., syringes) disclosed herein may therefore be able to administer higher possible concentrations of species present in a first fluid [e.g., doses of pharmaceutical composition (e.g., active agent(s) therein)] than would be possible by dilution.

In some embodiments, a device comprises or is a syringe or an injector (e.g., an auto-injector). In some embodiments, a syringe is manually operable [e.g., can be used to deliver fluid from an outlet of the syringe (e.g., an outlet of a needle) using manually applied force]. In some embodiments, an injector is manually operable [e.g., can be used to deliver fluid from an outlet of the injector (e.g., an outlet of a needle) using manually applied force]. In some embodiments, a manually applied force at which fluid flow occurs through a channel (e.g., in a device, such as a syringe or injector) is no more than 40 N (e.g., no more than 35 N, no more than 30 N, no more than 25 N, no more than 20 N, no more than 15 N, no more than 12 N, or no more than 10 N).

FIG. 2 panel A is a showing core annular flow injection of a viscous pharmaceutical composition (in this example a biologic) through a channel (e.g., hub and/or needle), which is lubricated by its buffer solution. Panel B illustrates enables drastically lower forces needed to be applied to at least a first fluid (e.g., viscous pharmaceutical composition) to cause flow using a syringe disclosed herein as compared to conventional syringe injections. Even at relatively low first fluid viscosities (e.g., about 10 cP, about 20 cP), the force that would need to be applied to a conventional syringe (e.g., by pinching the syringe to push a plunger of the syringe) exceeds what is achievable manually (the transition in shading indicates the manually feasible to manually infeasible transition). In contrast, even orders of magnitude larger viscosity fluids can be easily flowed via manually applied force when using devices (e.g., syringe) disclosed herein. Gray dots correspond to experimentally measured injection forces for fluids of different viscosities ranging from about 50 cP to about 1250 cP. Error bars for 49 cP fluid, 77 cP fluid, and 1266 cP fluid samples are on the order of the symbol size and are thus not shown. Conventional injection force is calculated for a 1 cm diameter syringe plunger via the Hagen-Poiseuille equation (eq. 1+2), with a ½″ 27G needle at 6 mL/min. 49 cP fluid, 77 cP fluid, and 500 cP fluid injections were performed at 6 mL/min with 12% v/v lubricant through a ½″ 27G needle. The 1266 cP fluid injection was performed at 7 mL/min with 22% v/v lubricant through a ½″ 27G thin wall needle. Panels C-D illustrate force profiles over time for a 500 cP fluid core annular flow injection (panel C) and a 1266 cP fluid core annular flow injection (panel D). As can be seen from panels C-D, while force varies over time, it stays well within the limit of manual injectability. For example, it would seem relatively easy to a user (e.g., physician or nurse) to pinch a syringe sufficiently hard to cause an about 6-7 mL/min flow to occur.

$$F = Δ P * A \qquad ( 1 )$$

$$Δ P = \frac{8 μ L Q}{π {R}^{4}} \qquad ( 2 )$$

Equations 1 and 2 can be used to calculate the expected injection force F for a conventional syringe and needle, given the pressure drop through the needle ΔP, the syringe plunger area A, the liquid viscosity u, the needle length L, the liquid flow rate Q, and the needle radius R.

Compatibility of pharmaceutical compositions with devices [e.g., syringes or injectors (e.g., auto-injectors)] disclosed herein is straightforward, as the devices do not qualitatively alter or interact with the pharmaceutical composition. Moreover, as discussed subsequently, relatively quite small amounts of lubricant and/or benign lubricants (e.g., that are immiscible with a pharmaceutical composition but nonetheless biocompatible or that are the same as a solvent and/or buffer used in a pharmaceutical composition) can be used while maintaining operability. This represents a marked advantage over other approaches to deliver (e.g., via syringe or injector) high doses of pharmaceutical compositions, like encapsulation and hyaluronidase, which require costly validation or reformulation to ensure compatibility.

Mitigation of viscous displacement and eccentricity are two important aspects which can facilitate injectability of fluids having a high viscosity, including viscosity in excess of 1,200 cP.

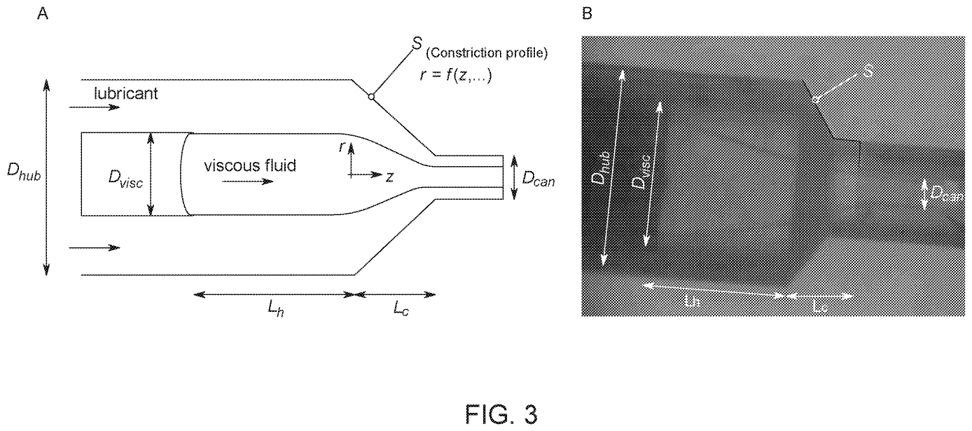

FIG. 3 denotes relevant physical channel (e.g., hub) design parameters. Relevant relevant fluidic parameters are the flow rates (Qᵢ and Qₒ), viscosities (μᵢ and μₒ), and densities (ρᵢ and ρₒ) of the inner first fluid and outer lubricant, respectively.

FIG. 3 panel A illustrates a diagram of a portion of a channel (e.g., in a hub) for initiating core annular flow with important dimensions denoted. The channel comprises a constriction. The constriction profile is drawn as a line, but can be any range of curved or piecewise functions, as discussed further subsequently. FIG. 3 panel B is an image of a fabricated hub with a stable core annular flow (visible by differentially dyed viscous first fluid).

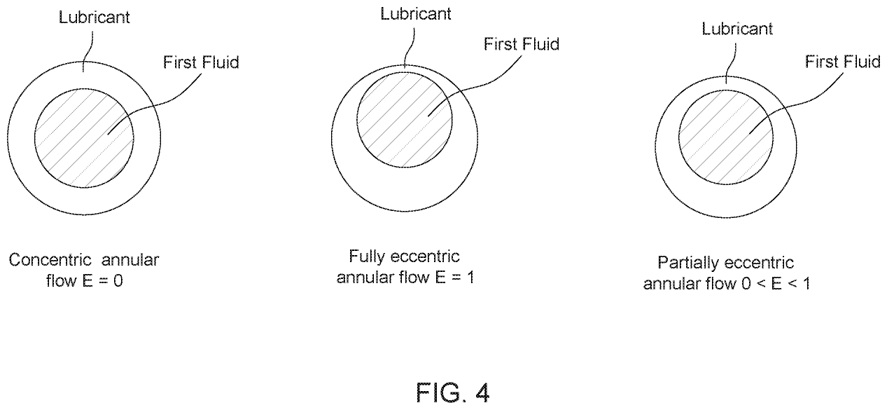

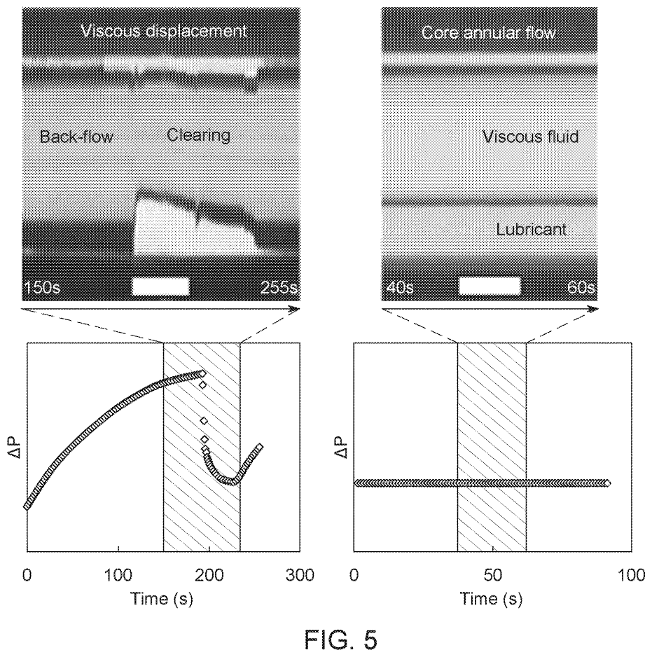

A stable core annular flow can be maintained by mitigation of various fluidic instabilities, such as, for example, high eccentricity and/or viscous displacement. Simultaneous flow through a channel of two fluids where one fluid, a lubricant, separates another fluid, a first fluid, from an interior surface of a channel can be characterized by an eccentricity parameter (E). Stable core annular flow is characterized by a low eccentricity parameter. In some embodiments, 0≤E<1. FIG. 4 illustrates characteristics of different eccentricity parameter regimes. Relevant discussion of such an eccentricity parameter can be found in Jayaprakash, V. et al., Enhancing the Injectability of High Concentration Drug Formulations Using Core Annular Flows, Adv. Healthcare Mater., 9 (18): 2001022 (2020), https://doi.org/10.1002/adhm.202001022, the disclosure of which is hereby incorporated by reference herein in its entirety. The closer E is to 0 (e.g., E<0.05, E<0.02, or E=0), the more benefit will be achieved from core annular flow. Nonetheless, significant benefit can still be realized at E closer to 1 (e.g., when E<1). Devices disclosed herein can achieve such eccentricity parameter values. It is preferable that a stable core annular flow be free from any instabilities in pressure drop or force required to maintain a constant injection rate (e.g., in some embodiments, a stable core annular flow has at most minimal instabilities caused by pressure drop or force required to maintain a constant injection rate). Significant variation in force or pressure drop is indicative of viscous displacement, or other fluidic instabilities. FIG. 5 illustrates viscous displacement as compared to stable core annular flow.

A difference in density between inner first fluid and outer lubricant can cause the inner and outer fluid to become eccentric if a device (e.g., syringe) is not directly aligned with gravity. This can lead to poor performance as previously described.

To mitigate eccentricity, fluids should be transported through any section of a channel in a smaller time than the time taken for the eccentricity to develop. Thus, two timescales can be defined: the timescale of convection (t_{c}) and the timescale of eccentricity (tₑ)

$${t}_{c} = \frac{L}{\overline{V}} = \frac{L {A}_{c}}{{Q}_{i} + {Q}_{o}} \qquad ( 3 )$$

$${t}_{e} = \sqrt{\frac{2 s}{\vert g cos θ ( 1 - \frac{{ρ}_{i}}{{ρ}_{o}} ) \vert}} \qquad ( 4 )$$

Where L is the device section length, A_{c} is the cross-sectional area, V̅ is the average flow velocity, s is the difference between the lubricant radius and the viscous liquid thread radius (i.e., the distance the viscous liquid must travel before it contacts the inner walls of the device), g is the gravitational constant, and θ is the angle of inclination from the horizontal.

Thus,

$$\frac{{t}_{c}}{{t}_{e}} < 1$$

suggests a criteria for designing devices (e.g., syringes) with core annular flow that properly mitigate eccentricity.

Notwithstanding this criteria, it has been observed that stable core annular flow can exist even when

$$\frac{{t}_{c}}{{t}_{e}} > 1 .$$

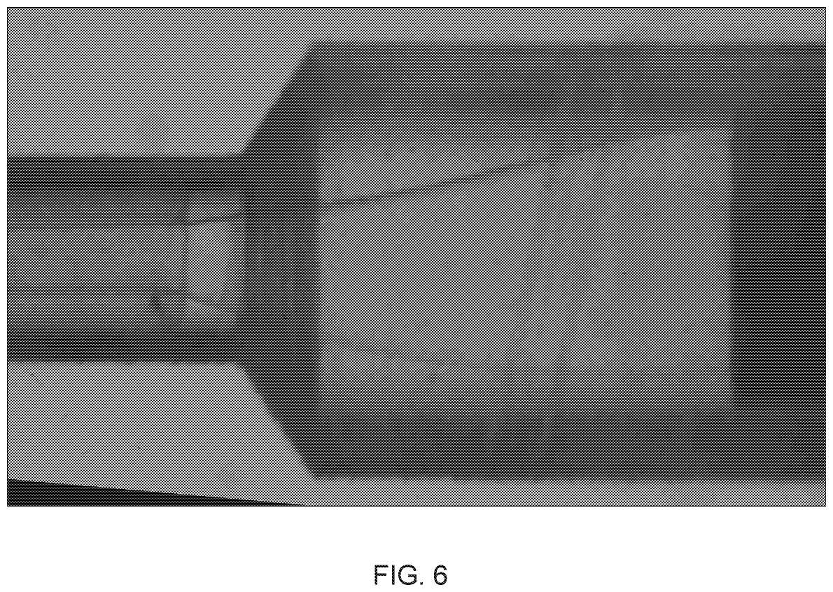

For example, FIG. 6 shows stable core annular flow for a prototype hub where

$$\frac{{t}_{c}}{{t}_{e}} = 3.7 .$$

While the (darker) first fluid (which is more viscous than surrounding lubricant) does fall in the constriction section (e.g., in a hub), fluidic effects near the constriction stabilize the flow and re-establish concentricity of the viscous fluid into and in a needle. The flow rate of first (inner) fluid is approximately double the flow rate of lubricant (outer fluid). Without wishing to be bound by any particular theory, it is expected that fluidic boundary and edge effects affect the development of eccentricity. Because the above equations (3-4) do not account for such effects, it is reasonable that stable core annular flow may still be observed even when

$$\frac{{t}_{c}}{{t}_{e}} > 1 .$$

Viscous displacement, as shown briefly in FIG. 5, is an instability which occurs when viscous fluid invades passage of a lubricant. This reduces/eliminates the lubricating effect of the lubricant, causing the fluidic resistance to increase sharply, effectively stopping/slowing the flow of inner and outer fluids. An inner viscous liquid may displace a lubricant and touchdown on the inner walls of a channel (e.g., comprised in a needle and/or hub) during viscous displacement. Viscous displacement is an undesirable instability which can serve as a (e.g., the) limiting factor in performance of core annular flow for high viscosities and for low volume fractions of lubricant.

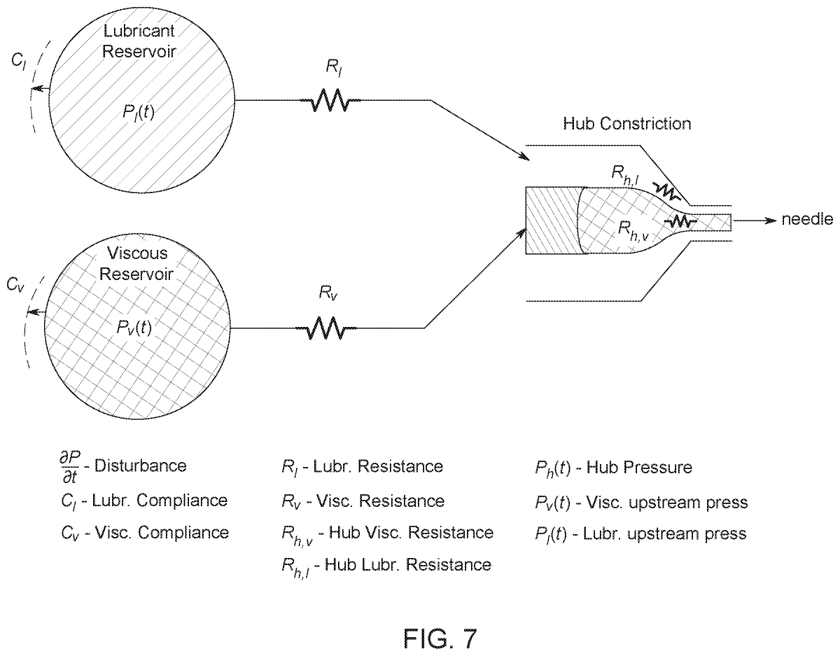

The laws of mass conservation can be applied to a control volume to a hub to derive a representative fundamental, representative equation which explains many aspects of viscous displacement at least for a channel (e.g., hub) design as indicated in FIG. 7. (Note that the following derivation while representative and useful in understanding the phenomenon of viscous displacement is not meant to very precisely characterize fluidic performance. That is, there may be some deviation between performance that would be anticipated from the following equations and that would be realized experimentally.) FIG. 7 illustrates elements considered in this analysis. It is assumed for simplicity that fluid is incompressible, so that the analysis can be simplified to a volume conservation equation:

$${Q}_{i n} = \frac{\partial V}{\partial t} + {Q}_{out} .$$

In the above volume conservation equation, Qᵢₙ is the volumetric flow rate of fluid into a control volume,

$$\frac{\partial V}{\partial t}$$

is the change in volume (V) of the control volume with time, and Qₒᵤₜ is the flow rate of fluid out of the control volume. Generally, the flow rates Qᵢₙ and Qₒᵤₜ are a controlled by relative pressures and fluidic resistances. For example, for laminar flow through a tube of circular cross section, the Hagen-Poiseuille equation reads:

$$Q = Δ P / R , where R = \frac{π {D}^{4}}{128 μL}$$

Here, R is the fluidic resistance, ΔP is the relevant pressure drop, D is the tube diameter, μ is the fluid viscosity, and L is the tube length.

With reference to FIG. 7, relevant pressures and fluidic resistances can be used to find representative expressions for Qᵢₙ and Qₒᵤₜ for the lubricant and the viscous fluid.

$${Q}_{i n , lubricant} = \frac{{P}_{l} ( t ) - {P}_{h} ( t )}{{R}_{l}}$$

$${Q}_{out , lubricant} = \frac{{P}_{h} ( t )}{{R}_{h , l}}$$

$${Q}_{i n , viscous} = \frac{{P}_{v} ( t ) - {P}_{h} ( t )}{{R}_{v}}$$

$${Q}_{out , viscous} = \frac{{P}_{h} ( t )}{{R}_{h , v}}$$

where a gage pressure relative to the pressure at the outlet of the control volume is assumed.

To determine a representative expression for the

$$\frac{\partial V}{\partial t}$$

term, mechanical compliance is considered. As pressure inevitably fluctuates by a non-zero amount in a device

$$( \frac{\partial V}{\partial t} ) ,$$

volume of fluidic pathways will oscillate due to mechanical deformation. For the example of a cylindrical tube of length L and initial, unpressurized radius of Rᵢ, its volume when it is subjected to a certain steady state pressure can be written as:

$$V = L \cdot ( π {R}_{i}^{2} + 2 π R Δ R )$$

Using Young's law and the formula for hoop stress, the increase in radius ΔR due to pressurization can be written as:

$$Δ R = \frac{P ( t ) {R}_{i}^{2}}{E δ}$$

where P(t) is the pressure, E is the Young's Modulus of the material, and δ is the tube thickness. By combining the two above equations and taking the partial derivative with respect to time (since this is a stability analysis), we get the following expression:

$$\frac{\partial V}{\partial t} = \frac{2 π {LR}_{i}^{3}}{E δ} \frac{\partial P}{\partial t}$$

More generally, the volume change term in response to a pressure fluctuation can be written as

$$\frac{\partial V}{\partial t} = C \frac{\partial P}{\partial t} ,$$

where C is the compliance of the control volume.

Using expressions for the volume change and flow rates in and out for the control volume gives the following representative equations:

$$General formulation : {Q}_{i n} = \frac{\partial V}{\partial t} + {Q}_{out}$$

$$Lubricant flow in the channel ( e . g . , hub ) : \frac{{P}_{l} ( t ) - {P}_{h} ( t )}{{R}_{l}} ∼ \frac{\partial P}{\partial t} {C}_{l} + \frac{{P}_{h} ( t )}{{R}_{h , l}}$$

$$Viscous flow in the channel ( e . g . , hub ) : \frac{{P}_{v} ( t ) - {P}_{h} ( t )}{{R}_{v}} ∼ \frac{\partial P}{\partial t} {C}_{v} + \frac{{P}_{h} ( t )}{{R}_{h , ν}}$$

Device design strategies for mitigating viscous displacement can be directly extracted from these two equations above. It should be noted that, without wishing to be bound by any particular theory, viscous displacement occurs at least when viscous fluid preferentially flows into the fluidic pathways of a lubricant, displacing the lubricant and decreasing its flow rate through a channel (e.g., hub) relative to the viscous fluid. Thus, device designs which balance the above equations such that adequate amounts of lubricant and viscous fluid flow through the channel (e.g., hub) in good relative ratio, in the face of destabilizing

$$\frac{\partial P}{\partial t}$$

perturbations can (e.g., will) mitigate viscous displacement.

There are two important flows at play: the Qₒᵤₜ flow (referred to hereafter as the “throughput flow”) and the flow associated with the

$$\frac{\partial V}{\partial t}$$

term (referred to hereafter as the “compliance flow”). When the throughput flow exceeds the compliance flow in regions of core annular flow, stable core-annular flow without viscous displacement can be achieved. Forming a ratio between the throughput flow and the compliance flow yields a nondimensional number:

$$S = \frac{{\frac{\partial P}{\partial z}}_{exit}}{\frac{\partial P}{\partial t} {CR}_{exit}}$$

where the C is the compliance of the control volume in question and the throughput flow is described in terms of a local pressure gradient at the exit and a resistance Rₑₓᵢₜ per unit length. It is desirable that this number is maximized above a critical threshold number at which viscous displacement occurs due to the depletion of lubricant to promote stable core annular flow. In seeking to maximize this number, it is desirable to minimize disturbances (∂P/∂t), minimize compliance (C), and minimize the resistance of core annular flow (Rₑₓᵢₜ).

Minimizing compliance will be considered first. As described in the above equations, unwanted destabilizing “compliance flows” which disturb the flow into and out of a channel (e.g., hub) are a result of the non-zero compliance of a device (e.g., channel and/or other component thereof), which leads to volume changes when subjected to pressure fluctuations

$$( {Q}_{c o m p l i a n c e} ∼ \frac{\partial P}{\partial t} \cdot C ) .$$

Compliance can come from a variety of sources, for example radial deformation of cylindrical passageways, axial deformation of compressed elements, and compressibility of fluids (liquid or gas).

FIG. 8 illustrates a device that exhibits core annular flow through a channel. The device includes the channel comprised at least partially in a hub. The hub connects two fluid reservoirs, one for viscous fluid and one for lubricant. The hub includes two inlets, each in fluid communication with one of the reservoirs, as well as a constriction (not separately illustrated) to induce core annular flow. Pressure can be applied to both reservoirs simultaneously, and optionally substantially equally, (as shown) in order for viscous fluid and lubricant to flow into and through the hub and then into and out of a needle in fluid communication with the hub on an opposite side of the hub from the fluid reservoirs. Core annular flow is established in the hub during fluid flow, due at least in part to a constriction in the hub, and persists through the needle. Various compliance terms that could be considered in device design (e.g., channel or channel constriction design) are shown, with contributions to compliance (compliance mechanisms) for the viscous fluid, Cᵥ, given on the left and for the lubricant, C_{cv}, given on the right. At right, expressions for estimating the magnitude of physical compliance are provided for a variety of compliance mechanisms, along with order-of-magnitude estimations of the compliances for relevant device (e.g., injector) physical parameters.

When optimizing a system with many compliant components, it is most pressing to first minimize the largest compliance, as it will have the most significant effect on the system. In the case of the device shown in FIG. 8, the compliance of the actuating finger is neglected, as its compliance effects both the lubricant and viscous sides, so its effects are more complex, and may be offset. The next highest compliance is that of air. It has been shown experimentally that the existence of air bubbles within the lubricant passageways significantly exacerbates viscous displacement. Operationally, this can be easily avoided by ensuring the lubricant passageways have a reduced presence of air bubbles, preferably are free of air bubbles.

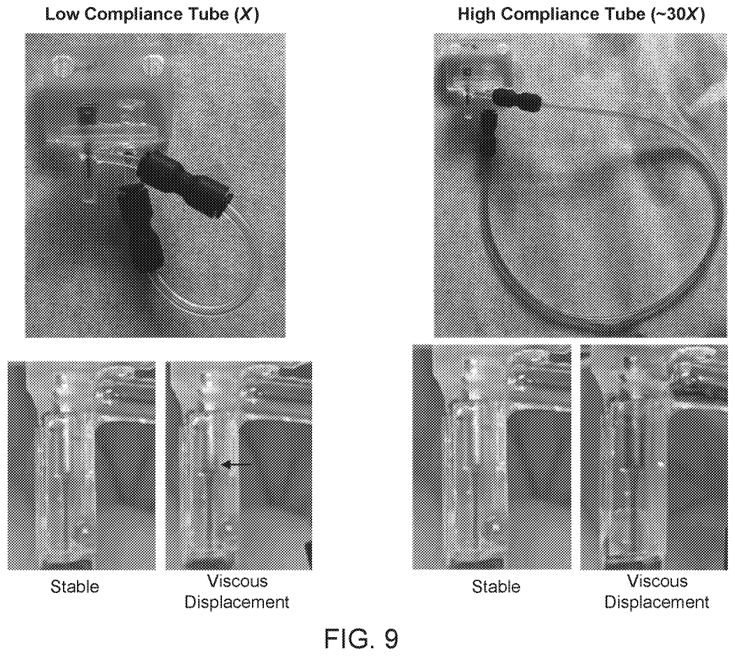

In order to demonstrate this concept more rigorously, a core annular flow hub which has a tunable compliance was tested, as shown in FIG. 9. Upon filming operation of the see-through hub, it was observed that when the hub has a lower tube compliance, it infrequently undergoes viscous displacement, and to only a small extent. Viscous displacement here is visible as the viscous inner fluid (dyed red) moves upwards into the lubricant passageway, as shown in FIG. 9. When operated at the same relative flow rates and with the same fluids, but a higher compliance tube, the hub exhibits drastically worse behavior, undergoing more frequent viscous displacement. The effects of viscous displacement are also more severe, as the volume of viscous fluid which invades the lubricant passageways is noticeably larger. The high compliance tube had a ~30× higher compliance due to both its greater length and the lower Young's Modulus of its material. Stable operation is characterized by core annular flow, wherein the lubricant fluid (clear) surrounds the viscous fluid (red) as it constricts. When viscous displacement occurs, the viscous fluid invades the lubricant passageway, as shown, for illustration purposes, by the black arrow on the left.

Minimizing the compliance of the system serves to lessen the compliance flows due to pressure disturbances, stabilizing the device operation. Compliance is sufficiently minimized when the compliance flows

$$( the \frac{\partial P}{\partial t} C term )$$

are small compared to the influx and outflux terms of the governing equations for lubricant flow and viscous flow above.

Various structural features in a channel and/or device may be used to minimize compliance and therefore mitigate viscous displacement or the potential for viscous displacement. For example, mechanisms for elimination of air bubbles in a device may be used, such as aerophobic surface chemistry (e.g., coatings) and use of thin fluid passageways which are less likely to entrap bubbles. Similarly, methods may be employed to remove dissolved air from fluid prior to flowing through a channel (e.g., prior to being provided to a reservoir (e.g., of a syringe or injector) or after being so provided and before flowing the fluid(s) from the reservoir) in order to decrease the likelihood of detrimental bubble formation. Numerical compliance can be defined as

$$C = \frac{\partial V}{\partial P}$$

(change in volume per change in pressure), so it has units of

$$[ \frac{{s}^{2} \cdot {m}^{4}}{k g} ] .$$

High compliance elements having a compliance of at least

$$3 \cdot {10}^{- 12} [ \frac{{s}^{2} \cdot {m}^{4}}{k g} ]$$

were observed to have poor performance. Additionally, an air bubble of 0.1 mL volume, having a compliance of

$$1 \cdot {10}^{- 12} [ \frac{{s}^{2} \cdot {m}^{4}}{k g} ] ,$$

has been observed to cause poor performance. A typical polypropylene syringe is

$$3 \cdot {10}^{- 13} [ \frac{{s}^{2} \cdot {m}^{4}}{k g} ] ,$$

which has been observed to have good performance. Thus, in some embodiments, compliance is preferably <10⁻¹¹, more preferably <10⁻¹², even more preferably <5*10⁻¹³.

If a lubricant reservoir, or any lubricant fluidic pathway upstream of a channel (e.g., hub) has significant mechanical compliance, fluctuations in device pressure could lead to substantial changes in flow rates into and out of the channel (e.g., hub). If the pressure increases substantially, the fluidic pathways will increase in volume, resulting in a smaller flow of lubricant into and out of the channel (e.g., hub). This will lead to the depletion of lubricant within the channel (e.g., hub), causing expansion of the viscous fluid in the channel (e.g., hub), leading to a viscous displacement event.

Matching upstream resistance will be considered next. Looking to FIG. 7, we can assume that pressure of lubricant in a channel (e.g., hub) and pressure of viscous fluid in the channel are the same (Pₕ) due to parallel streamlines. We can thus estimate the upstream pressures in both the lubricant reservoir and the viscous reservoir as follows:

Pₗ(t)−Pₕ(t)=Qₗ·Rₗ

Pᵥ(t)−Pₕ(t)=Qᵥ·Rᵥ

where Qₗ and Qᵥ are the flow rates of lubricant and viscous fluid, respectively, and Rₗ and Rᵥ are the fluidic resistances of the lubricant and viscous passageways, respectively. Neglecting compliance effects, we can express the relative flow rates of lubricant and viscous fluid into the hub as:

$$\frac{{Q}_{l}}{{Q}_{v}} = \frac{{R}_{v}}{{R}_{l}} \cdot \frac{{P}_{l} ( t ) - {P}_{h} ( t )}{{P}_{v} ( t ) - {P}_{h} ( t )}$$

To promote stability of the lubricated core annular flow, it is important that relative flow rate of lubricant and viscous fluid stay stable to avoid depletion of the lubricant. In this instability analysis, we assume that the pressure within the hub may change rapidly over a timescale for which the bulk lubricant pressure does not change, therefore, we assume that Pₗ(t) and Pᵥ(t) are constant in time.

In this expression, if Pₗ≠Pᵥ, then

$$\frac{{Q}_{l}}{{Q}_{v}}$$

will be a function of Pₕ (t). However, if Pₗ=Pᵥ, then the expression becomes only a function of the fluidic resistances, wherein

$$\frac{{Q}_{l}}{{Q}_{v}} = \frac{{R}_{v}}{{R}_{l}} .$$

This is a highly favorable case for stability, as the relative flow rates are independent of time. In essence, pressure perturbations in the system may affect the absolute flow rates of lubricant and viscous, but do not affect the relative flow rates, as the flow rates are equally affected.

Such a condition can be engineered by choosing appropriate resistances given the desired relative flow rate of lubricant and viscous fluid, such that pressure fluctuations minimally affect the stability.

Increasing lubricant resistance can also have the effect of resisting backwards flow, which may occur during viscous displacement, limiting the extent to which viscous fluid can invade backwards into lubricant passageways in a channel and lessening the deleterious impact of viscous displacement.

A prototype device was fabricated incorporating these resistance matching concepts, and it significantly outperformed prototypes which did not include these resistance matching concepts. Device with resistance matching exhibited smooth injection of 1266 cP fluid without noticeable viscous displacement, with a relative flow rate of lubricant to viscous fluid of 0.14. For a device without resistance matching, injection of 1266 cP fluid at a relative flow rate of lubricant to viscous fluid of 0.27 is not possible without significant viscous displacement which disrupts the injection.

Fluctuations in pressure in a device can change the amount of each fluid flowing into and out of a channel (e.g., hub). If pressure fluctuations change the flow rates of viscous fluid and lubricant in substantially different ways, the flow may become destabilized. For example, suppose the pressure within the channel (e.g., hub) increases substantially, and assume also that the pressure in reservoir(s) for the viscous fluid and lubricant is constant. This will reduce the pressure drop between the fluid reservoir(s) and the channel (e.g., hub), reducing the flow rate of both lubricant and viscous fluid into the channel (e.g., hub). The change in flow rate of the lubricant will be a function of the lubricant fluidic resistance, Rₗ and the upstream pressure Pₗ. The change in flow rate of the viscous fluid will be a function of the viscous fluidic resistance, Ry, and the upstream pressure Pᵥ. If the fluidic resistances of the viscous fluidic pathway and lubricant fluidic pathway are not properly correlated, the change in flow rates between the two fluids will be substantially different. This could lead to a depletion of lubricant in the channel (e.g., hub), causing viscous displacement. Appropriately correlating the fluidic resistances can minimize the effects of viscous displacement.

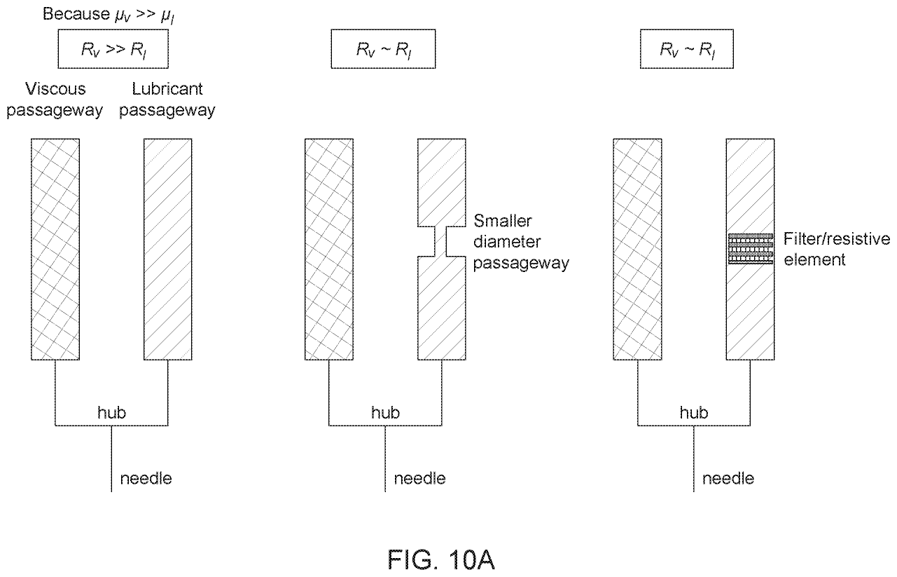

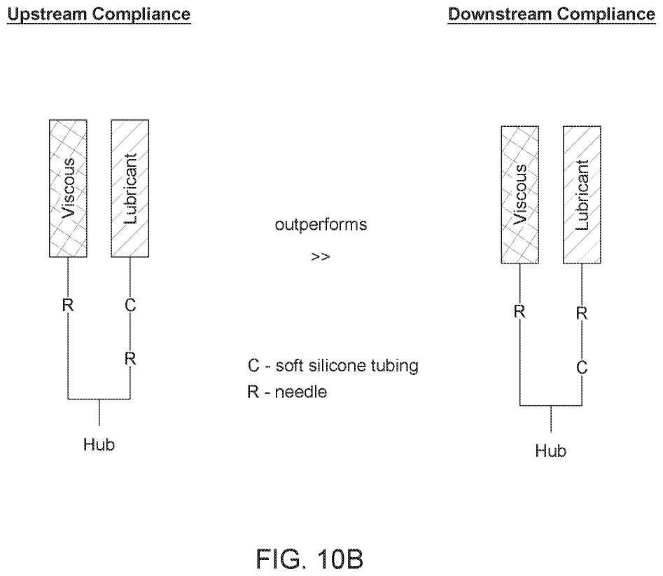

One way to do this is to have a small “choke point” within the lubricant passageway, as it will likely be difficult to manufacture a device which matches the resistance with a constant-diameter passageway. This specific resistive region could be in the form of a thin tube, or also in the form of a resistive element [e.g., filter (e.g., porous membrane) and/or densely packed elements with small pores]. These two approaches could also be used in combination. FIG. 10A illustrates both a choke point (small diameter passageway), in the middle drawing, and a resistive element (e.g., filter). A small diameter passageway (choke point) in a reservoir, for example for a lubricant, may have, for example, a diameter that is no more than 50%, no more than 40%, no more than 30%, no more than 20%, or no more than 10% of a larger diameter of the reservoir. A filter may have a pore size and pore density such that a total cross sectional area of the pores (e.g., averaged over a thickness of the filter) through which fluid can flow in a reservoir is no more than 50%, no more than 40%, no more than 30%, no more than 20%, or no more than 10% of a diameter of the reservoir. Different diameters of a reservoir for a viscous fluid and of a reservoir for a lubricant may be used to at least partially resistance match flow of the two fluids (e.g., may be used in combination with a small diameter passageway and/or a resistive element (e.g., filter)). Diameter in these cases refers to internal diameter since thickness of reservoirs, or other fluid passageways, may vary, for example, for compliance minimization reasons.

In general to employ this concept, resistance of lubricant must typically be increased, because viscosity of the lubricant is generally much lower than of a viscous fluid. It is unlikely that a constant-diameter passageway can be used for a lubricant passageway because of manufacturing constraints at small diameters, at least without being prohibitively expensive. Instead, it is more likely that this resistance could be provided with a short section of a smaller diameter passageway, or by using a resistive element (e.g., a filter).

Resistance can be defined as ΔP_{upstream,l}=Rₗ·Qᵢ, where ΔPᵤₚₛₜᵣₑₐₘ refers to the pressure drop between a fluid reservoir and initiation of core annular flow in a channel (e.g., in a hub). The subscript l refers to the lubricant. Thus, similarly, ΔP_{upstream,v}=Rᵥ·Qᵥ·Rᵥ and Rₗ are preferably correlated given the relative flow rates of viscous fluid and lubricant such that ΔP_{upstream,v} and ΔP_{upstream,l} are not too different. Suitable performance has been observed at at

$$\frac{Δ {P}_{upstream} , v}{Δ {P}_{upstream} , l} \sim 10 , 000$$

even when a device was completely unoptimized for resistance matching. Substantially better performance may be observed when

$$\frac{{R}_{v}}{{R}_{l}} ≅ 1 .$$

In some embodiments,

$$\frac{Δ {P}_{upstream} , v}{Δ {P}_{upstream} , l}$$

is no more than 100,000 in a device, preferably no more than 10,000, more preferably no more than 1,000, no more than 100, no more than 10, or no more than 5. In some embodiments,

$$\frac{Δ {P}_{upstream} , v}{Δ {P}_{upstream} , l}$$

is in a range of from 0.5 to 5, e.g., of from 0.5 to 2, or of from 0.5 to 1.5. Such pressure drops may be used in a double barrel design or in a design with two fluid reservoirs that output into separate inlets into a hub, such as in FIG. 8.

Minimization of disturbances will be considered next. Reducing the magnitude of disturbances helps to stabilize flow through devices. Pressure disturbances contribute to destabilizing the relative flow rates of lubricant and viscous fluids, which can lead to viscous displacement. Disturbances can come from a variety of sources, such as non-constant actuation force/pressure, environmental vibrations, non-uniformity of fluidic properties (density, viscosity, etc.), particulate matter, and others.

Thus it is preferable (and improves stability performance) to take precautions with the injection process and fluids such as: reducing fluctuations in actuation profile; ensuring uniformity of fluids (well-mixed, uniform temperature, etc.); removal of particulates which may disturb the flow profile of core annular flow; reducing external vibrations; or a combination thereof.

Fluctuations in actuation force/pressure can lead to instability. Use of a single pressure headspace which is shared between the lubricant and viscous reservoirs can mitigate instabilities arising for this reason. In this way, fluctuations in pressure affect both the lubricant and the viscous fluid similarly, keeping relative flow rates similar. The resistances can be designed to secure a desired relative flow rate of viscous fluid to lubricant when applying pressure from a single pressure headspace. Highly stable operation of such a device for a viscous fluid viscosity of 500 cP and a relative flow rate of lubricant to viscous fluid of 0.33 has been demonstrated.

Pressure fluctuations can be particularly damaging when they couple with high-compliance components, causing compliance flows. Another strategy which could be used to minimize the negative effects of disturbances is the use of dampers to isolate sensitive, high-compliance components. This concept is illustrated in FIG. 10B. Viscous flow inherently damps high-frequency pressure disturbances, as the re-arrangement of the velocity profile during flow through a passageway consumes energy. Viscous flow does not immediately adjust to changes in pressure gradient. The frequency above which disturbances are effectively damped is described by the characteristic timescale of viscous communication:

$${t}_{s} ∼ \frac{ρ {D}^{2}}{μ} , f = 1 / {t}_{s}$$

Thus, given the diameter of the tube (D) and the fluid density (ρ) and the fluid viscosity (μ), a damper can be designed to isolate a compliant component from a particular frequency range, lessening the effects of compliant flow and decreasing the severity of viscous displacement.

It has been observed that designs which isolated the most compliant components from a channel (e.g., hub) with a damper (having high fluidic resistance) significantly outperformed designs which did not use an isolating damper.

Given that viscous displacement is a transient instability effect, minimizing the disturbances to a device will increase the stability of the device. Minimizing pressure fluctuations will minimize the ∂V/∂t terms in the above equations, which can lead to a more constant influx and outflux of lubricant and viscous fluid into and out of a channel (e.g., hub), lessening lubricant depletion effects.

Structural features of a device that can minimize disturbances include: shared pressure headspace; a shared actuation system (e.g., where a viscous fluid and lubricant do not necessarily have a shared headspace but rather are pushed by the same force); regions with high viscous losses can be used to damp pressure disturbances; and reduced diameter passageway that is sufficiently small to incur viscous losses; and use of dampers.

The frequencies which are damped are a function of the diameter.

$$( {f}_{d a m p e d} > \frac{μ}{ρ {R}^{2}} ) ,$$

where R is the radius of the circular cross section tube. A fluidic damper could have the same design as what is shown in FIG. 10A. For example, a fluidic damper could be a region of smaller diameter, or a resistive element (e.g., filter) in which viscous losses are significant. It has been experimentally observed that the relative placement of these elements has significant effects. In some embodiments, a damper is placed between a hub (where core annular flow is initiated) and a (e.g., the most) compliant element upstream of the hub. Such arrangements can limit pressure disturbances experienced by that compliant element, reducing likelihood of viscous displacement. This damping effect has been observed for a smaller passageway diameter of 0.2 mm, so it may be preferable to have a passageway diameter of <2.0 mm (e.g., <1.0 mm, <0.5 mm, or <0.2 mm).

Minimizing channel resistance will be considered next. Analytical constriction profile optimization may be performed by computing a profile for which an expression for the shear is held constant through the constriction, from Dₕ to D_{can}. Such a profile should evenly constrict the fluid flow.

$$0 = μ ( \frac{\partial }{\partial r} ( \frac{1}{r} \frac{\partial }{\partial r} ( r {v}_{r} ) ) + \frac{{\partial }^{2} {v}_{r}}{\partial {z}^{2}} ) - \frac{\partial P}{\partial r} \qquad ( 5 )$$

$$0 = μ ( \frac{1}{r} \frac{\partial }{\partial r} ( r \frac{\partial {v}_{z}}{\partial r} ) + \frac{{\partial }^{2} {v}_{z}}{\partial {z}^{2}} ) - \frac{\partial P}{\partial z} \qquad ( 6 )$$

The Navier-Stokes equation in axisymmetric cylindrical coordinates is written above in the viscous limit in the r and z directions. Gravity is neglected. Through scaling and integration of individual shear terms, analytical hub profiles can be determined which keep that specific shear term constant.

For example, the term for shear in the z direction due to changing v_{z} can be integrated as follows:

$$μ ( \frac{{\partial }^{2} {v}_{z}}{\partial {z}^{2}} ) = 0 \qquad ( 7 )$$

$${v}_{z} = {C}_{1} z + {C}_{2} \qquad ( 8 )$$

$$\overline{{v}_{z}} ( z ) = \frac{{Q}_{i}}{π {R ( z )}^{2}} \qquad ( 9 )$$

$$R ( z ) = {( \frac{\frac{{Q}_{i}}{π}}{{C}_{1} z + {C}_{2}} )}^{1 / 2} \qquad ( 10 )$$

Where R(z) forms the profile of a constriction, for example as shown in FIG. 3. Other conditions result in alternative profiles; for example specifying a constant dA_{c}/dz results in an exponential profile.

Across all four shear terms shown in the Navier-Stokes equation, there are possible contributions to analytical profiles of the order of

$$R ( z ) = f ( {z}^{\frac{- 1}{3}} , {z}^{\frac{- 1}{2}} , {z}^{- 1} , {z}^{1} , {z}^{2} , {z}^{3} )$$

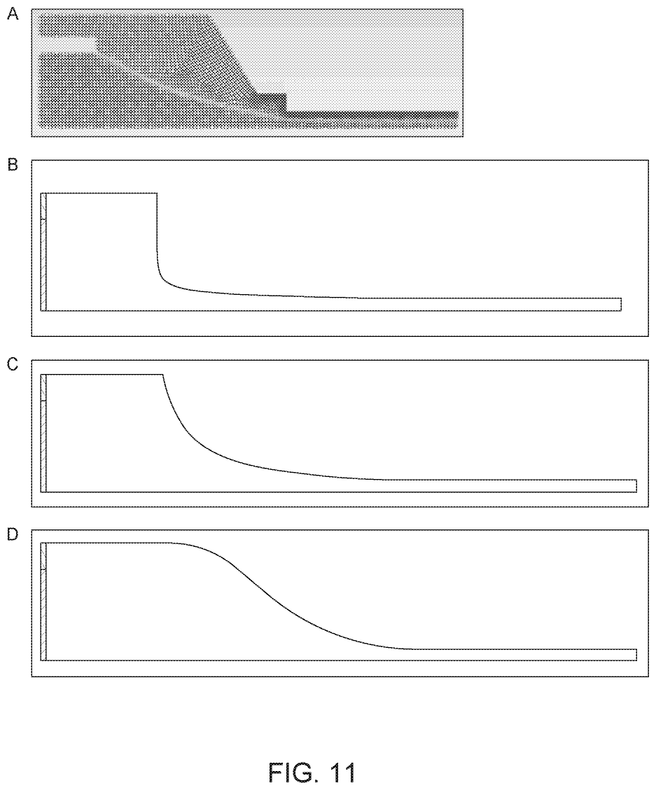

A simulation model was constructed in Ansys Fluent software to aid hub profile optimization. This model computes the laminar flow profile for two fluids in a hub, constriction, and needle as shown in FIG. 11. Panel A illustrates simulated core annular flow for an experimental hub geometry in accordance with FIG. 3 panel B. Blue (on bottom) indicates the inner viscous first fluid (or inlet), while red (on top) indicates the presence of the lubricant (or inlet). As can be seen, lubricant fills in sharp corners. Panels B-D illustrate simulated hub profiles following analytical equations of the form R(z)=(C₁z+C₂)ⁿ with n as −1/3, −1, and +3, respectively. In some embodiments, a constriction has an interior profile having a shape defined by R(z)=(C₁z+C₂)ⁿ where −1≤n≤−0.1 (e.g., n=−1/3).

In some embodiments, geometries which best mitigate viscous displacement should minimize fluidic stress in a (e.g., any) constriction (e.g., that is in a hub). Thus, the metrics used to compare the simulated hub profiles are maximum fluid strain and pressure gradients in the axial and radial direction at various points in a syringe. Selected values are shown in Table 1.

TABLE 1

| | Maximum dP/dx | Maximum dP/dy | Maximum strain |
|---|---|---|---|
| n = +3 | −1.30E07 | 1.77E07 | 2.3E05 |
| n = −1 | −7.30E06 | 7.14E06 | 1.6E05 |
| n = −⅓ | −4.50E06 | 3.16E06 | 1.3E05 |

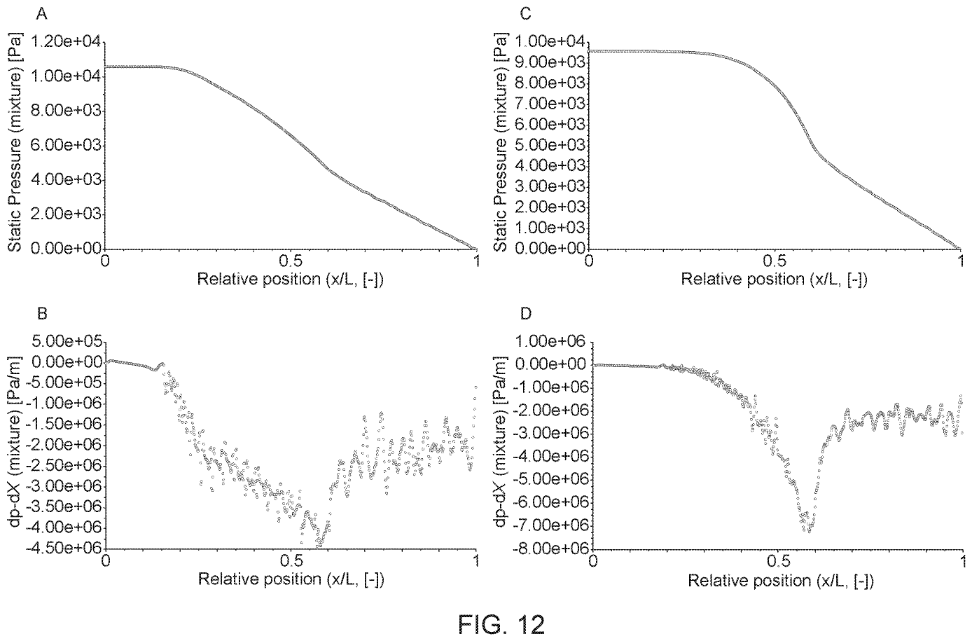

From Table 1, the constriction profile with n=−1/3 (illustrated in FIG. 11 panel B) minimizes fluidic shear compared to the other two hub profiles illustrated (n=−1, n=+3). This brings about the realization that constriction at smaller diameters can be more important than constriction at larger diameters: the n=−1/3 profile emphasizes a gradual taper when diameter of a constriction is small, and a sharper taper when the diameter is larger. In general, convex hub profiles outperform concave hub profiles. This effect can be seen when comparing the plots of pressure along the axis for n=3 and n=−1/3 constrictions (e.g., in hubs) as shown in FIG. 12. The n=−1/3 hub (panels A-B) demonstrate a relatively consistent constriction, with the pressure gradient remaining roughly constant throughout. However, an n=3 constriction (e.g., in a hub) (panels C-D) has an initially small pressure gradient at the beginning of the constriction that develops into a very large pressure gradient at the end of the constriction. The relatively faster taper of the profile near the neck leads to increased pressure gradients and fluidic shear at the neck for the n=3 constriction, suggesting it will be more susceptible to viscous displacement.

In a general sense, each individual fluid element which is flowing into a hub with a constriction (e.g., each reservoir flowing into the hub) will either proceed through a constriction or will participate in “compliance flow” (the ∂V/∂t), i.e. flow elsewhere other than through the constriction. One can associate a “stiffness” with each of these kinds of flows, which are correlated to the hub fluidic resistance and the system compliance, respectively. If the mechanical compliance of a device is very low (i.e., the device is very stiff) when compared to the stiffness of the fluidic flow through the constriction, there will be minimal compliance flow, as the fluid chooses the path of least resistance(s) and flows through the constriction. Such a thought framework leads to the conclusion that the resistance of the constriction (and therefore resistance to core annular flow) should be minimized. In this way, both lubricant and viscous fluid can preferentially flow into and out of a hub, sustaining lubrication and avoiding viscous displacement.

Minimizing core annular flow resistance can also lead to reduced pressure drops and thus a lower actuation force of the device, which is additionally preferable.

The resistance of a constriction in a channel (and therefore to core annular flow) can be expressed in more depth than a single number. Because of the transient nature of viscous displacement, it is helpful to consider how constriction and/or core annular flow resistance changes in response to slight perturbations. Consider first the operation of a device initially at steady state, which is then perturbed slightly. Should this perturbation cause lubricant flow to decrease relative to viscous flow (whether due to compliance or due to fluidic resistance), the lubrication effect of the core annular flow will be less effective. This will cause an increase in channel (e.g., hub) pressure required to drive flow. This increase in channel pressure could have the effect of further decreasing the lubricant flow, which further increases pressure, leading to a run-away transient effect of lubricant depletion and viscous displacement. The rate at which the constriction and/or core annular flow resistance increases for a given perturbation is a critical quality in this situation: designs which are relatively resistant to perturbations (e.g., have small changes in flow resistance or fluid flow rates when subjected to perturbations) are much more likely to remain stable than those which undergo significant changes. In this sense, stiffness of a constriction (and/or fluid undergoing core annular flow) can be useful to consider not only under steady state operation, but also in response to perturbations.

These transient considerations can be especially important during device startup, for example, where hub does not initially contain the viscous fluid. Upon flow initiation, a large plug of viscous fluid enters a hub, and must be constricted into the needle. This is associated with a large, sudden increase in pressure, which can lead to a run-away compliance flow which depletes the lubricant as described above, leading to viscous displacement. Preloading a hub and/or pausing between hub loading and further fluid flow may therefore be desirable to mitigate such deleterious effects.

The foregoing analysis is not constrained to only the components used above as examples, such an analysis can be performed for any relevant fluidic pathways or mechanical components of a device disclosed herein, for example whether similar or dissimilar to the two reservoir syringe illustrated in FIG. 8. In other words, resistance and compliance of any and all fluidic pathways in the device can be considered, and may be desirably considered, in the analysis of a device and may contribute to fluidic performance.

In some embodiments, a constriction (e.g., in a hub) comprises a convex portion relative to an interior of a channel (e.g., through the hub) (e.g., a portion of which is comprised in the hub). For example, in some embodiments, a second derivative of an interior profile of a constriction is positive (>0) at at least some portion of the profile (e.g., using the coordinate system in FIG. 3 panel A). In some embodiments, a constriction (e.g., in a hub) is convex relative to an interior of a channel (e.g., through the hub) at at least some portion of the constriction. In some such embodiments, a constriction (e.g., in a hub) is entirely convex relative to an interior of a channel (e.g., through the hub) (e.g., a portion of which is comprised in the hub). For example, in some embodiments, a second derivative of an interior profile of a constriction is positive (>0) at every point along the profile. In some embodiments, a constriction (e.g., in a hub) tapers relatively more sharply with position at a larger diameter end of the constriction and relatively less sharply with position at a smaller diameter end.

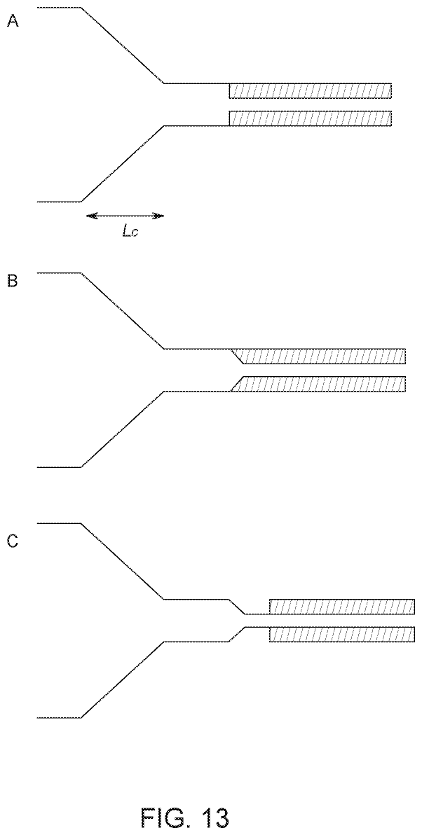

A device (e.g., syringe or injector) may comprise a needle and a hub. The needle and the hub may be in fluid communication with each other. A needle may comprise an outlet of a channel. A body of a needle may define a portion of a channel (e.g., a lumen of the needle may define the portion). A portion of a channel may be comprised in a needle. An interface between a hub and a (e.g., inserted) needle may also produce improved performance by using a more gradual taper that at least partially mitigates viscous displacement. As, in some embodiments, a needle has a smallest internal diameter (e.g., of a lumen of the needle) of an entire device, introduction of core annular flow into the needle can be quite critical to maintaining performance (e.g., achieving flow of a first fluid out of an outlet of a channel that is an outlet of the needle or flow of a sufficiently high rate). Generally, sudden constriction at the needle-hub interface should be avoided. FIG. 13 compares a conventional needle-hub interface (panel A) to alternative needle-hub interfaces (panels B-C) that mitigate or eliminate issues caused by sudden constriction present in conventional needle-hub interfaces. The illustrations in FIG. 13 are in cross section, with hubs with simple (linear) constrictions shown in light shading and a needle shown in dark shading.

In some embodiments, a portion of the needle is disposed inside (e.g., an end of) a hub, for example as illustrated in FIG. 13 panels A-B. Unlike the conventional design (panel A), a first alternative needle-hub interface (panel B) does not include a sharp constriction. A portion of the needle disposed inside a hub may comprise a needle inlet that comprises a gradual constriction to an inner diameter of the needle, for example as illustrated in FIG. 13 panel B. A gradual constriction may be bevel, for example in a range of 15-75 degrees, such as a range of from 15 to 60 degrees or from 15 to 45 degrees. In another alternative needle-hub interface design, an outlet of the hub is disposed inside of a needle (panel C). A blunt-ended needle can be inserted without the need for needle machining, but still avoiding a sharp needle-hub interface and associated viscous displacement issues. In some embodiments, a hub comprises a second constriction (e.g., comprising a convex portion) in order to facilitate achieving a sufficiently small smallest hub diameter to be inserted into a needle. Such needles, whether disposed partially inside or partially outside (e.g., around) a hub outlet may be held in place by pressure and/or friction and/or by adhesive (e.g., glue). In some embodiments, a needle is smaller than 22 gauge (e.g., 25 gauge or 27 gauge) (e.g., has a gauge in a range from 23 gauge to 27 gauge). In some embodiments, a needle is a 23 gauge needle, a 24 gauge needle, a 25 gauge needle, a 26 gauge needle, a 27 gauge needle, a 30 gauge needle, or smaller.

Two additional geometric optimization studies have been carried out (and incorporated in the highest-performing prototype as described below in Example 1). First, distance between a channel inlet (e.g., in a hub), for a first fluid, and the beginning of a constriction (e.g., in a hub) (Lₕ) can have a significant effect on viscous displacement behavior in addition to being an important parameter for eccentricity mitigation. Operating conditions and/or constriction profile (e.g., shape) may influence preferred Lₕ. In some embodiments, an inlet and a constriction are disposed no more than 5 mm apart, for example no more than 4 mm, no more than 3 mm, no more than 2 mm, no more than 1 mm, no more than 0.8 mm, no more than 0.6 mm, no more than 0.5 mm, or no more than 0.4 mm apart.

Second, relative area ratio between a first fluid inlet (Aᵢ) and a lubricant inlet (Aₒ) has been investigated. Aᵢ and Aₒ in this instance are defined by the following equations.

$${A}_{i} = \frac{π}{4} {D}_{v i s c}^{2} \qquad ( 11 )$$

$${A}_{o} = \frac{π}{4} ( {D}_{h u b}^{2} - {D}_{v i s c}^{2} ) \qquad ( 12 )$$

In some embodiments, Aᵢ/Aₒ is preferably in a range of from 0.07 to 1.38. Higher Aᵢ/Aₒ ratios may yield better performance with respect to decreasing viscous displacement, though extremely high Aᵢ/Aₒ become difficult to fabricate while maintaining concentricity of subsequent flow. In some embodiments, a channel comprises two inlets, one for a first fluid and one for a lubricant. For example, double barrel syringes (e.g., with an inner and outer barrel) have such inlets. Two reservoir devices, such as the one illustrated in FIG. 8, are other examples of such inlets. In some embodiments, a ratio of area of an inlet, for a first fluid, to area of a second inlet, for a lubricant, is in a range of from 0.02 to 5. For example, the ratio of areas may be in a range of from 0.02 to 2, from 0.02 to 4, from 0.02 to 3, e.g., from 0.04 to 2, from 0.04 to 4, from 0.04 to 3, from 0.05 to 2, from 0.06 to 2, from 0.07 to 2, from 0.02 to 4, from 0.02 to 3, from 0.02 to 1.8, from 0.02 to 1.6, from 0.02 to 1.4, from 0.04 to 4, from 0.04 to 3, from 0.04 to 1.8, from 0.04 to 1.6, from 0.04 to 1.4, from 0.06 to 4, from 0.06 to 3, from 0.06 to 2, from 0.06 to 1.8, from 0.06 to 1.6, from 0.06 to 1.4, from 0.07 to 4, from 0.07 to 3, from 0.07 to 2, from 0.07 to 1.8, from 0.07 to 1.6, from 0.07 to 1.4, from 0.1 to 4, from 0.1 to 3, from 0.1 to 2, from 0.1 to 1.8, from 0.1 to 1.6, from 0.1 to 1.4, from 0.2 to 4, from 0.2 to 3, from 0.2 to 2, from 0.3 to 4, from 0.3 to 3, from 0.3 to 2, from 0.4 to 4, from 0.4 to 3, from 0.4 to 2, from 0.5 to 4, from 0.5 to 3, from 0.5 to 2, from 0.5 to 1.8, from 0.5 to 1.6, or from 0.5 to 1.4.

In some embodiments, a lubricant comprises water. For example, a lubricant may be or comprise a water-based solution (e.g., saline). In some embodiments, a lubricant comprises a biocompatible and/or metabolizable oil (e.g., squalene, safflower oil, a fluorinated oil (e.g., HFE-7500), mineral oil, and/or triglyceride oil). In some embodiments, a lubricant comprises a buffer (e.g., a pharmaceutically acceptable buffer, such as a buffer suitable for use with a biologic). In some embodiments, a lubricant comprises a formulation (e.g., a pharmaceutical formulation, such as a biologic formulation). In some embodiments, a lubricant comprises an immunologic adjuvant (e.g., MF59, AS02, AS03 and/or AS04). In some embodiments, a lubricant is aqueous. In some embodiments, a lubricant is non-aqueous. In some embodiments, a lubricant comprises a buffer. In some embodiments, a first fluid comprises a buffer that is the same as a buffer comprised in a lubricant.

In some embodiments, a first fluid and a lubricant comprise mutually distinct components. For example, a first fluid may comprise a pharmaceutical composition comprising an aqueous solvent (e.g., water), while a lubricant is non-aqueous (e.g., comprises an organic solvent). In some embodiments, a first fluid (e.g., pharmaceutical composition) and a lubricant comprise one or more components (e.g., a solvent and/or a buffer) that are the same. For example, in certain embodiments, a lubricant and a first fluid (e.g., pharmaceutical composition) comprise water. In certain embodiments, a first fluid and a lubricant comprise one or more components that are different and one or more components that are the same. For example, in some embodiments, a first fluid and a lubricant comprise the same components except that a first fluid comprises a pharmaceutical composition (e.g., a biologic). For example, in certain embodiments, a first fluid and a lubricant both comprise water and the first fluid further comprises a pharmaceutical composition (e.g., a biologic) and the lubricant does not. In some embodiments, a first fluid and a lubricant comprise exactly the same components (e.g., a buffer) except that one of the fluids (e.g., a first fluid) has an additional component (e.g., a pharmaceutical composition). In some embodiments, a first fluid and a lubricant are miscible. In some embodiments, a first fluid and a lubricant are immiscible.

In some embodiments, a lubricant and first fluid do not mix substantially in at least a portion of a device (e.g., in a needle, hub, barrel, or combination thereof). In some embodiments, a lubricant mixes with a first fluid at most 50% while in at least a portion of a device (e.g., in a needle, hub, barrel, or combination thereof). That is, at most 50% (e.g., at most 40%, at most 30%, at most 20%, at most 10%, or at most 5%) of a lubricant is mixed with a first fluid while in at least a portion of a device (e.g., in a needle, hub, barrel, or combination thereof) while the remainder of the lubricant remains unmixed with the first fluid. In some embodiments, extent of mixing may be determined by visual inspection and/or measuring concentration changes.

In some embodiments, a first fluid and a lubricant comprise exactly the same components (e.g., a buffer and a drug), but the concentrations of one or more of the components are different (e.g., a drug). For example, in some embodiments, a first fluid and a lubricant comprise exactly the same components (e.g., a buffer and a pharmaceutical composition), but concentration of one or more of the components (e.g., a pharmaceutical composition) is higher in a first fluid. In some embodiments, a first fluid has a high concentration of a biologic and a lubricant has a low (e.g., no) concentration of the biologic, but the first fluid and lubricant are otherwise identical, viscosity and/or density of the first fluid may be higher (e.g., much higher) than that of the lubricant.

In some embodiments, molar concentration of one component (e.g., a pharmaceutical composition) in a lubricant is greater than or equal to 5%, greater than or equal to 10%, greater than or equal to 20%, greater than or equal to 30%, greater than or equal to 40%, greater than or equal to 50%, greater than or equal to 60%, greater than or equal to 75%, greater than or equal to 90%, or greater than or equal to 95% less than molar concentration of that component in a first fluid. In some embodiments, the molar concentration of one component (e.g., a pharmaceutical composition) in a lubricant is less than or equal to 100%, less than or equal to 99%, less than or equal to 95%, less than or equal to 90%, less than or equal to 80%, less than or equal to 70%, less than or equal to 60%, or less than or equal to 50% molar concentration of that component in a first fluid. Combinations of these ranges are also possible (e.g., greater than or equal to 5% and less than or equal to 100%, or greater than or equal to 10% and less than or equal to 50%). For example, if molar concentration of the component was 1M in a first fluid and 0.1M in a lubricant, the molar concentration of the component in the lubricant would be 90% less than that in the first fluid.

In some embodiments, lubricant is no more than 50 vol % (e.g., no more than 40 vol %, no more than 30 vol %, no more than 25 vol %, no more than 20 vol %, no more than 15 vol %, no more than 10 vol %, or no more than 5 vol %) of total volume of material inside a channel. In some embodiments, a volume ratio of a first fluid to a lubricant is no less than no less than 1:1 (e.g., no less than 2:1, no less than 3:1, no less than 4:1, no less than 5:1, no less than 6:1, no less than 7:1, no less than 8:1, or no less than 9:1). In some embodiments, a volume ratio of first fluid to lubricant is no more than 200:1 (e.g., no more than 100:1).

In some embodiments, a first fluid [e.g., in a channel (e.g., flowing through a channel)] has a viscosity in a range of from 5 cP to 10,000 cP. For example, a first fluid may have a viscosity in a range of from 5 cP to 5,000 cP, from 5 cP to 4,000 cP, from 5 cP to 3,000 cP, from 5 cP to 2,500 cP, from 5 cP to 2,000 cP, from 5 cP to 1,750 cP, from 5 cP to 1,500 cP, or from 5 cP to 1,250 cP. For example, a first fluid may have a viscosity in a range of from 10 cP to 10,000, from 10 cP to 5,000 cP, from 10 cP to 4,000 cP, from 10 cP to 3,000 cP, from 10 cP to 2,500 cP, from 10 cP to 2,000 cP, from 10 cP to 1,750 cP, from 10 cP to 1,500 cP, or from 10 cP to 1,250 cP. For example, a first fluid may have a viscosity in a range of from 15 cP to 10,000, from 15 cP to 5,000 cP, from 15 cP to 4,000 cP, from 15 cP to 3,000 cP, from 15 cP to 2,500 cP, from 15 cP to 2,000 cP, from 15 cP to 1,750 cP, from 15 cP to 1,500 cP, or from 15 cP to 1,250 cP. For example, a first fluid may have a viscosity in a range of from 20 cP to 10,000, from 20 cP to 5,000 cP, from 20 cP to 4,000 cP, from 20 cP to 3,000 cP, from 20 cP to 2,500 cP, from 20 cP to 2,000 cP, from 20 cP to 1,750 cP, from 20 cP to 1,500 cP, or from 20 cP to 1,250 cP. For example, a first fluid may have a viscosity in a range of from 50 cP to 10,000, from 50 cP to 5,000 cP, from 50 cP to 4,000 cP, from 50 cP to 3,000 cP, from 50 cP to 2,500 cP, from 50 cP to 2,000 cP, from 50 cP to 1,750 cP, from 50 cP to 1,500 cP, or from 50 cP to 1,250 cP. For example, a first fluid may have a viscosity in a range of from 75 cP to 10,000, from 75 cP to 5,000 cP, from 75 cP to 4,000 cP, from 75 cP to 3,000 cP, from 75 cP to 2,500 cP, from 75 cP to 2,000 cP, from 75 cP to 1,750 cP, from 75 cP to 1,500 cP, or from 75 cP to 1,250 cP. For example, a first fluid may have a viscosity in a range of from 100 cP to 10,000, from 100 cP to 5,000 cP, from 100 cP to 4,000 cP, from 100 cP to 3,000 cP, from 100 cP to 2,500 cP, from 100 cP to 2,000 cP, from 100 cP to 1,750 cP, from 100 cP to 1,500 cP, or from 100 cP to 1,250 cP. For example, a first fluid may have a viscosity in a range of from 250 cP to 10,000, from 250 cP to 5,000 cP, from 250 cP to 4,000 cP, from 250 cP to 3,000 cP, from 250 cP to 2,500 cP, from 250 cP to 2,000 cP, from 250 cP to 1,750 cP, from 250 cP to 1,500 cP, or from 250 cP to 1,250 cP. For example, a first fluid may have a viscosity in a range of from 500 cP to 10,000, from 500 cP to 5,000 cP, from 500 cP to 4,000 cP, from 500 cP to 3,000 cP, from 500 cP to 2,500 cP, from 500 cP to 2,000 cP, from 500 cP to 1,750 cP, from 500 cP to 1,500 cP, or from 500 cP to 1,250 cP. For example, a first fluid may have a viscosity in a range of from 1,000 cP to 10,000, from 1,000 cP to 5,000 cP, from 1,000 cP to 4,000 cP, from 1,000 cP to 3,000 cP, from 1,000 cP to 2,500 cP, from 1,000 cP to 2,000 cP, from 1,000 cP to 1,750 cP, from 1,000 cP to 1,500 cP, or from 1,000 cP to 1,250 cP. In some embodiments, a first fluid [e.g., in a channel (e.g., flowing through a channel)] has a viscosity of at least 10 cP, (e.g., at least 15 cP, greater than 20 cP, at least 50 cP, at least 100 cP, at least 200 cP, at least 300 cP, at least 500 cP, at least 750 cP, at least 1,000 cP, at least 1,100 cP, at least 1,200 cP, at least 1,300 cP, at least 1,500 cP, at least 2,000 cP, at least 3,000 cP, at least 4,000 cP, or at least 5,000 cP.

In some embodiments, a channel is sized and shaped such that fluid having viscosity anywhere in a range of at least from 5 cP to 10,000 cP can flow through the channel (e.g., from an inlet to and through an outlet) [e.g., under core annular flow (e.g., coaxial lubrication)]. In some embodiments, a channel is sized and shaped such that fluid having viscosity anywhere in a range of at least from 5 cP to 5,000 cP can flow through the channel. In some embodiments, a channel has a curvature such that fluid having viscosity anywhere in a range of at least from 5 cP to 10,000 cP can flow through the channel (e.g., from an inlet to and through an outlet) [e.g., under core annular flow (e.g., coaxial lubrication)]. In some embodiments, a channel has a curvature such that fluid having viscosity anywhere in a range of at least from 5 cP to 5,000 cP can flow through the channel.

In some embodiments, a lubricant has a viscosity in a range of from 0.5 cP to 10 cP. For example, in some embodiments, a lubricant has a viscosity in a range of from 0.5 cP to 5 cP or 0.5 cP to 2 cP. In some embodiments, a lubricant has a viscosity less than 10 cP, for example less than 5 cP, less than 4 cP, less than 3 cP, less than 2 cP, or less than 1.5 cP.

In some embodiments, a lubricant and a first fluid exhibit core annular flow (e.g., coaxial lubrication) (e.g., a stable core annular flow) while simultaneously flowing through a channel. In some embodiments, a lubricant and a first fluid exhibit core annular flow (e.g., coaxial lubrication) (e.g., a stable core annular flow) the first fluid flows through a device. A core annular flow (e.g., through a channel and/or device) may occur with an efficiency (η) greater than 20% (e.g., greater than 34%, greater than 40%, greater than 50%, greater than 52%, greater than 60%, greater than 70%, greater than 76%, greater than 80%, or greater than 90%). Such a core annular flow may occur when a longitudinal axis of a channel lies within 45 degrees of a line perpendicular to gravity. Such a core annular flow may occur with a miscible first fluid and lubricant. In some embodiments, a core annular flow (e.g., through a channel and/or device) occurs with an efficiency (η) greater than 40% (e.g., greater than 50%, greater than 52%, greater than 60%, greater than 70%, greater than 76%, greater than 80%, or greater than 90%) with an immiscible first fluid and lubricant. In some embodiments, a first fluid can flow with an efficiency (η) greater than 20% (e.g., greater than 34%, greater than 40%, greater than 50%, greater than 52%, greater than 60%, greater than 70%, greater than 76%, greater than 80%, or greater than 90%) when a longitudinal axis of the channel lies within 45 degrees of a line perpendicular to gravity and/or with a miscible first fluid and lubricant.

In some embodiments, a fluid (e.g., pharmaceutical composition) delivery device includes a channel (e.g., in a hub, optionally also a needle) extending from an inlet to an outlet, where the device has an efficiency (η) of fluid flow from the inlet to and through the outlet of greater than 20% (e.g., greater than 34%, greater than 40%, greater than 50%, greater than 52%, greater than 60%, greater than 70%, greater than 76%, greater than 80%, or greater than 90%) (e.g., under core annular flow). In some embodiments, In some embodiments, a fluid (e.g., pharmaceutical composition) delivery device includes a channel (e.g., in a hub, optionally also a needle) extending from an inlet to an outlet, where the device has an efficiency (η) of fluid flow from the inlet to and through the outlet of greater than 40% (e.g., greater than 50%, greater than 52%, greater than 60%, greater than 70%, greater than 76%, greater than 80%, or greater than 90%) with an immiscible first fluid and lubricant. In some embodiments, the first fluid has a viscosity of at least 5 cP, at least 10 cP, at least 15 cP, or greater than 20 cP. In some embodiments, a fluid (e.g., pharmaceutical composition) delivery device includes a channel (e.g., in a hub, optionally also a needle) extending from an inlet to an outlet, where the device has an efficiency (η) of fluid flow through the channel of greater than 20% (e.g., greater than 34%, greater than 40%, greater than 50%, greater than 52%, greater than 60%, greater than 70%, greater than 76%, greater than 80%, or greater than 90%) (e.g., under core annular flow). In some embodiments, the first fluid has a viscosity of at least 5 cP, at least 10 cP, at least 15 cP, or greater than 20 cP. In some embodiments, a fluid (e.g., pharmaceutical composition) delivery device has an efficiency (η) of fluid flow through an outlet of the device of greater than 20% (e.g., greater than 34%, greater than 40%, greater than 50%, greater than 52%, greater than 60%, greater than 70%, greater than 76%, greater than 80%, or greater than 90%) (e.g., under core annular flow). In some embodiments, the first fluid has a viscosity of at least 5 cP, at least 10 cP, at least 15 cP, or greater than 20 cP. In some embodiments, a first fluid can flow through a channel (e.g., of a device) with an efficiency (η) greater than 20% (e.g., greater than 34%, greater than 40%, greater than 50%, greater than 52%, greater than 60%, greater than 70%, greater than 76%, greater than 80%, or greater than 90%) (e.g., under core annular flow). In some embodiments, the first fluid has a viscosity of at least 5 cP, at least 10 cP, at least 15 cP, or greater than 20 cP.

In some embodiments, a longitudinal axis of the channel lies within 45 degrees of a line perpendicular to gravity. In some embodiments, a longitudinal axis of the channel lies within 45 degrees of a line perpendicular to gravity. In some embodiments, a first fluid can flow with an efficiency (η) greater than 20% (e.g., greater than 34%, greater than 40%, greater than 50%, greater than 52%, greater than 60%, greater than 70%, greater than 76%, greater than 80%, or greater than 90%) when a longitudinal axis of the channel lies within 45 degrees of a line perpendicular to gravity. In some embodiments, a first fluid can flow with an efficiency (η) greater than 20% (e.g., greater than 34%, greater than 40%, greater than 50%, greater than 52%, greater than 60%, greater than 70%, greater than 76%, greater than 80%, or greater than 90%) when flowed under core annular flow with a miscible lubricant.

An efficiency (η) of a channel can be expressed as a ratio of theoretical pressure required to drive flow through an outlet of a channel to actual pressure required to drive flow through the outlet. For example, an efficiency can be taken as a ratio of theoretical pressure required to drive the flow through an outlet of a channel (e.g., of a device) under laminar, ideal, fully developed, perfectly concentric core annular flow conditions (ΔP_{ideal}) to actual pressure required to drive the flow through an outlet of a channel (ΔP_{channel}):

$$η = \frac{Δ {P}_{ideal}}{Δ {P}_{channel}} .$$

An efficiency (η) can also be expressed as a ratio between flow rate of a first fluid through a channel (Q_{channel}) and ideal flow rate of a first fluid under core annular flow (Q_{ideal}) for a specified constant ΔP:

$$η = \frac{{Q}_{c h a n n e l}}{{Q}_{i d e a l}} .$$

An efficiency (η) of a core annular flow device can also be expressed as pressure reduction factors under ideal core annular flow and actual channel pressure reduction factor:

$$η = \frac{{R}_{c h a n n e l}}{{R}_{i d e a l}} .$$

An efficiency (η) of a device can be expressed as a ratio of theoretical pressure required to drive flow through an outlet of the device to actual pressure required to drive flow through the outlet. For example, an efficiency can be taken as a ratio of theoretical pressure required to drive the flow through an outlet of a device under laminar, ideal, fully developed, perfectly concentric core annular flow conditions (ΔP_{ideal}) to actual pressure required to drive the flow through the entire device (ΔP_{device})

$$η = \frac{Δ {P}_{i d e a l}}{Δ {P}_{d e v i c e}} .$$

An efficiency (η) can also be expressed as a ratio between flow rate of a first fluid through a device (Q_{device}) and ideal flow rate of a first fluid under core annular flow (Q_{ideal}) for a specified constant ΔP:

$$η = \frac{{Q}_{d e v i c e}}{{Q}_{i d e a l}} .$$

An efficiency (η) of a core annular flow device can also be expressed as pressure reduction factors under ideal core annular flow and actual device pressure reduction factor:

$$η = \frac{{R}_{d e v i c e}}{{R}_{i d e a l}} .$$

In some embodiments, a channel has a largest inner diameter (e.g., of a barrel) of no more than 5 cm, no more than 4 cm, no more than 3 cm, no more than 2 cm, or no more than 1 cm. In some embodiments, a device has a largest inner diameter (e.g., of a barrel) of no more than 5 cm, no more than 4 cm, no more than 3 cm, no more than 2 cm, or no more than 1 cm. In some embodiments, a constriction has a largest inner diameter of no more than 5 cm, no more than 4 cm, no more than 3 cm, no more than 2 cm, no more than 1 cm, no more than 0.9 cm, no more than 0.8 cm, no more than 0.7 cm, no more than 0.6 cm, no more than 0.5 cm, no more than 0.4 cm, no more than 0.3 cm, no more than 0.2 cm, or no more than 0.1 cm. In some embodiments, a hub has a largest inner diameter of no more than 5 cm, no more than 4 cm, no more than 3 cm, no more than 2 cm, no more than 1 cm, no more than 0.9 cm, no more than 0.8 cm, no more than 0.7 cm, no more than 0.6 cm, no more than 0.5 cm, no more than 0.4 cm, no more than 0.3 cm, no more than 0.2 cm, or no more than 0.1 cm. In some embodiments, a largest inner diameter of a hub is the same as a largest inner diameter of a constriction. In some embodiments, a hub has a smallest inner diameter (e.g., at a hub-needle interface) (e.g., at an outlet of the hub) of no more than 5 cm, no more than 4 cm, no more than 3 cm, no more than 2 cm, no more than 1 cm, no more than 0.9 cm, no more than 0.8 cm, no more than 0.7 cm, no more than 0.6 cm, no more than 0.5 cm, no more than 0.4 cm, no more than 0.3 cm, no more than 0.2 cm, no more than 0.1 cm, no more than 0.9 mm, no more than 0.8 mm, no more than 0.7 mm, no more than 0.65 mm, no more than 0.6 mm, no more than 0.55 mm, no more than 0.5 mm, no more than 0.45 mm, or no more than 0.4 mm. In some embodiments, a needle has a smallest inner diameter (e.g., a needle lumen size) (e.g., at an outlet of the needle) of no more than 1 mm, no more than 0.9 mm, no more than 0.8 mm, no more than 0.7 mm, no more than 0.65 mm, no more than 0.6 mm, no more than 0.55 mm, no more than 0.5 mm, no more than 0.45 mm, or no more than 0.4 mm. In some embodiments, a barrel of a syringe has a smallest interior diameter (e.g., at a tip) of no more than 5 cm, no more than 4 cm, no more than 3 cm, no more than 2 cm, no more than 1 cm, no more than 0.9 cm, no more than 0.8 cm, no more than 0.7 cm, no more than 0.6 cm, no more than 0.5 cm, no more than 0.4 cm, no more than 0.3 cm, no more than 0.2 cm, or no more than 0.1 cm.

In some embodiments, a channel comprises two or more separate constrictions. In some embodiments, a hub comprises two or more separate constrictions. In some embodiments, two adjacent constrictions are separated along a channel (e.g., in a hub or in a barrel) by no more than 5 mm, no more than 4 mm, no more than 3 mm, no more than 2 mm, no more than 1 mm, or no more than 0.5 mm. In some embodiments, a device or apparatus comprises two or more constrictions along a channel comprised therein. For example, in some embodiments, a hub comprises two constrictions. As another example, in some embodiments, a hub comprises a first constriction and a needle comprises a second constriction. In some embodiments, a barrel comprises a first constriction (e.g., at a tip of the barrel) and a hub comprises a second constriction. In some embodiments, a barrel comprises two constrictions. Exemplary constrictions in accordance with some embodiments are discussed herein with respect to a constriction in a hub but are not necessarily limited as being located in such a hub.

In some embodiments, a hub comprises an inlet for a channel. In some embodiments, a hub comprises an outlet for a channel. In some embodiments, a needle comprises an outlet for channel. In some embodiments, a needle comprises an inlet for a channel. In some embodiments, a barrel comprises an inlet for a channel. In some embodiments, a hub comprises two inlets, for example one for a first fluid and one for a lubricant. In some embodiments, a barrel comprises two inlets, for example one for a first fluid and one for a lubricant, for example at a tip of the barrel. In some embodiments, a hub is attached to (e.g., glued to or compressed into or onto) a needle.

As used herein, the term “pharmaceutical composition” refers to a composition in which an active agent is formulated together with one or more pharmaceutically acceptable carriers. A pharmaceutical composition (e.g., formulation) may comprise a small molecule and/or a biologic. A pharmaceutical composition may comprise a monoclonal antibody (mAb), a polyclonal antibody (pAb), an enzyme, a peptide, a recombinant therapeutic protein formulation, or a combination thereof. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in liquid form. A pharmaceutical composition may be a concentrated formulation where one or more active ingredients [e.g., one or more antibodies (e.g., comprising one or more monoclonal antibodies and/or one or more polyclonal antibodies), enzymes, peptides, proteins, or combinations thereof] are present at high concentration, for example at least 25 mg/mL (of solvent, e.g., water), at least 30 mg/mL, at least 40 mg/mL, at least 50 mg/mL, at least 60 mg/mL, at least 70 mg/mL, at least 75 mg/mL, at least 80 mg/mL, at least 90 mg/mL, at least 100 mg/mL, at least 125 mg/mL, at least 150 mg/mL, at least 175 mg/mL, or at least 200 mg/mL (e.g., and no more than 500 mg/mL). A pharmaceutical composition may be formulated for parenteral administration, for example by subcutaneous, intramuscular, intravenous or epidural injection, for example using a syringe or injector (e.g., auto-injector). A pharmaceutical composition may be formulated as, for example, a sterile solution or suspension. A pharmaceutical composition may be formulated for transdermal administration (e.g., using a patch comprising one or more needles). Pharmaceutical compositions disclosed herein are typically fluids. A first fluid may be or comprise a pharmaceutical composition. A pharmaceutical composition may be referred to as a drug. A pharmaceutical composition may be or comprise a drug, a monoclonal antibody, an enzyme, a peptide, a recombinant therapeutic protein, a biologic, a bone putty, a hydrogel, cells, a biopharmaceutical, or a combination thereof. For example, in certain embodiments, a first fluid comprises a pharmaceutical composition that is or comprises a concentrated drug formulation (e.g., biologic).

### EXAMPLES

In order that the application may be more fully understood, the following examples are set forth. It should be understood that these examples are for illustrative purposes only and are not to be construed as limiting in any manner.

### Example 1

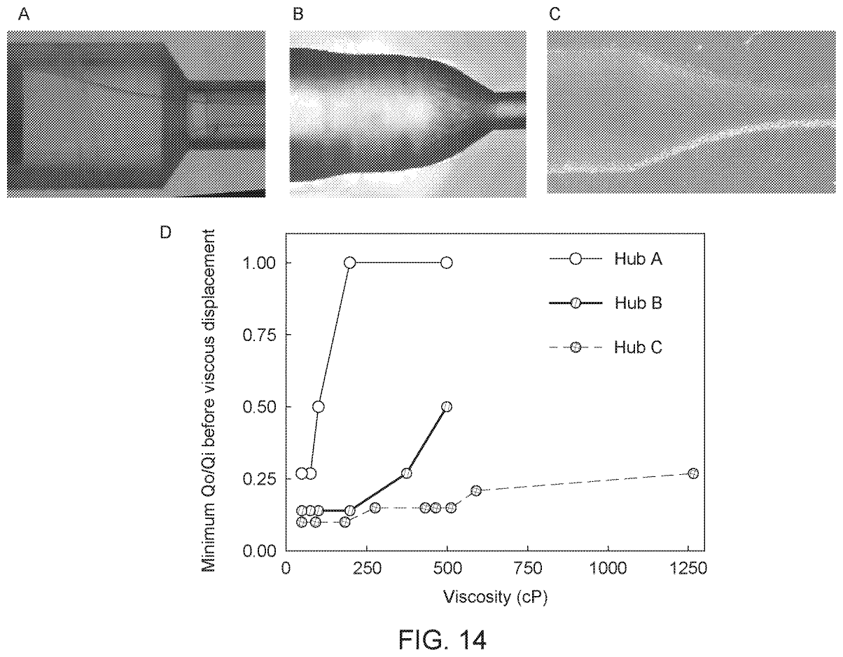

Numerous hubs having different profiles have been fabricated and performance tested. In line with simulations discussed above, it has been found that that convex hub profiles which taper more aggressively at large diameters before tapering less aggressively at small diameters perform best. FIG. 14 illustrates, in panels A-C, images of three selected hubs. A measure of their corresponding performance is plotted in panel D.

Panel A illustrates a hub with right angle discontinuities and rapid constriction. The hub was fabricated with standard drill bits. Panel B illustrates a hub with an extended taper at a more gradual angle, fabricated with custom modified drill bits. Panel C illustrates a hub with a convex exponential hub profile fabricated via 3D printing. The exponential hub profile in panel C most closely matches that of n=−1.

Panel D provides a plot of minimum Qₒ/Qᵢ possible without incurring viscous displacement for a range of viscosities, a measurement of performance of the hubs illustrated in panels A-C. Qₒ is flow rate of a (n outer) lubricant and Qᵢ is flow rate of a first (inner) fluid. with the minimum Qₒ/Qᵢ possible without incurring viscous displacement for a range of viscosities. Lower Qₒ/Qᵢ represents better performance. The exponential profile hub (panel C) significantly outperforms the other two hubs.

### Example 2

This section outlines details on calculating efficiency of a core annular flow in embodiments of the present disclosure. As shown in Table 2, the present disclosure presents core annular flows that require lower pressures for the same flow rate and viscosity conditions as compared to other designs.

Certain devices can generate core annular flow in a more efficient manner than others, for example, depending on the flow geometries before the joining of two fluids and/or during the constriction of the fluids. That is, even with the same hub outlet (needle) diameter and length, same fluid viscosities, and the same fluid flow rates, certain devices will have lower pressure requirements than others depending on how efficiently the flow can be constricted and how coaxial the flow is. Channel geometries disclosed herein (e.g., having certain curvature) are significantly more efficient than other designs, for example those in Table 2.

TABLE 2

| Work | Efficiency | Limiting conditions |
|---|---|---|
| Jayaprakash, V. et al., 2020 | 42-52% | Non-miscible buffer |
| Jayaprakash, V. et al., 2020 | 20% | |
| U.S. Pat. No. 9,737,662 | 34% | Perfectly vertical operation |
| PCT Application Publication | 18% | |
| No. WO 2013/114114 | | |
| Tested embodiments of | Up to at least 76% | None |
| present disclosure | | |

The efficiency (η) of a device can be expressed as the ratio between the theoretical pressure required to drive the flow through an outlet (e.g., of a needle) under laminar, ideal, fully developed, perfectly concentric core annular flow conditions (ΔP_{ideal}) and the actual pressure required to drive the flow through the entire device (ΔP_{device}):

$$η = \frac{Δ {P}_{i d e a l}}{Δ {P}_{d e v i c e}} \qquad ( 13 )$$

The value of ΔP_{device} can be measured with a pressure sensor, or by dividing the device injection force by the cross-sectional area of force application. Owing to a linear relationship between pressure drop and flow rate, the efficiency can also be defined as the ratio between flow rate of the first viscous fluid through the device (Q_{device}) and the ideal flow rate of the first viscous fluid under core annular flow (Q_{ideal}) for a specified constant ΔP:

$$η = \frac{{Q}_{d e v i c e}}{{Q}_{i d e a l}} \qquad ( 14 )$$

In both cases, an efficiency of 1 represents a perfectly efficient device whose pressure drop does not exceed that required for the core annular flow through an outlet (e.g., of a needle). Higher efficiencies are preferred to reduce actuation pressure, reduce injection times, or both.

The ideal relationship between flow rate and pressure drop can be calculated for a cylindrical geometry by considering the Navier Stokes equation in cylindrical coordinates under laminar conditions for both the first and second fluid:

$$0 = - \frac{\partial P}{\partial z} + \frac{{μ}_{i}}{r} \frac{\partial }{\partial r} ( r \frac{\partial {v}_{z}}{\partial r} ) 0 \leq r \leq {r}_{i} , \qquad ( 15 )$$

$$0 = - \frac{\partial P}{\partial z} + \frac{{μ}_{o}}{r} \frac{\partial }{\partial r} ( r \frac{\partial {v}_{z}}{\partial r} ) {r}_{i} \leq r \leq {r}_{o} , \qquad ( 16 )$$

where rᵢ is the radius of the inner first fluid, and rₒ is the radius of the cylindrical wall, e.g., the needle inner diameter. Here μᵢ and μₒ represent the inner and outer fluid viscosities and

$$\frac{\partial P}{\partial z}$$

is the pressure gradient along the axis of the cylindrical geometry. Assuming a constant pressure gradient, integrating the expressions and applying the appropriate boundary conditions (no slip at the wall, shear and velocity continuity at the interface of the two fluids, and symmetry) yields the following expressions for the flow rate of the inner first fluid and the outer second fluid under core annular flow:

$${Q}_{i , CAF} = \frac{π {r}_{i}^{4}}{8} \frac{d {P}_{C A F}}{d z} ( \frac{2}{{μ}_{o}} ( 1 - \frac{{r}_{o}^{2}}{{r}_{i}^{2}} ) - \frac{1}{{μ}_{i}} ) \qquad ( 17 )$$

$${Q}_{o} = \frac{π}{8 {μ}_{o}} \frac{d {P}_{C A F}}{d z} ( {( {r}_{o}^{2} - {r}_{i}^{2} )}^{2} ) \qquad ( 18 )$$

These relations between the pressure drop and the flow rates represent the limiting ideal efficiency of 1.0, where

$${Q}_{i , CAF} = {Q}_{ideal} and \frac{Δ {P}_{i d e a l}}{L} = \frac{d {P}_{C A F}}{d z}$$

for some cylindrical length L.

Another more convenient method to calculate the efficiency is to compare the force reduction factor afforded by a core annular flow device compared to the ideal pressure reduction factor. The pressure reduction factor can be defined as:

$$R = \frac{Δ {P}_{no CAF}}{Δ {P}_{CAF device}} , \qquad ( 19 )$$

where ΔP_{no CAF} is the pressure drop for a conventional device which does not use core annular flow, and ΔP_{CAFdevice} is the pressure drop for a core annular flow device. This pressure reduction factor is considered under otherwise identical conditions, with the flow rate of the first inner fluid in core annular flow matching the flow rate of the first fluid in the device without core annular flow, matching needle diameters, lengths, and first fluid viscosities.

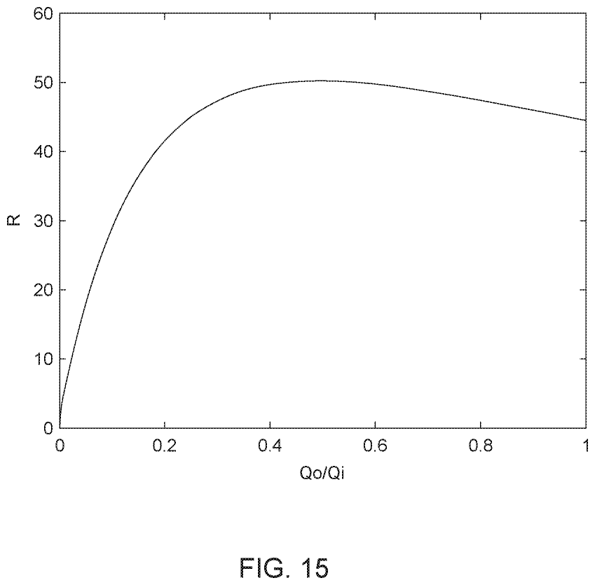

The pressure reduction factor under ideal conditions is shown in FIG. 15 as a function of Qₒ/Qᵢ for a first inner viscosity of 100 cP and a second outer viscosity of 1 cP.

The efficiency of a core annular flow device can thus be calculated by comparing the pressure reduction factors under ideal core annular flow and the actual device pressure reduction factor:

$$η = \frac{{R}_{d e v i c e}}{{R}_{i d e a l}} . \qquad ( 20 )$$

Calculated efficiency is a dimensionless number that applies to a range of combinations of force/pressure and flow rates for a given device. That is, a calculated efficiency characterizes a device (e.g., hub) at more than a particular flow rate and/or force/pressure. Nonetheless, in general, when calculating efficiency, one will either choose a force/pressure and measure a flow rate or choose a flow rate and measure the force/pressure required. The different formulae previously given for efficiency will have some minor natural variation but values calculated by the different methods are still comparable.

In the work of Jayaprakash, V. et al., 2020 on aqueous miscible lubricant, a pressure reduction coefficient of η≈2.6 was found. The ideal pressure reduction factor for these conditions is 12.75, resulting in the efficiency of 20% for a miscible (in this case aqueous) lubricant.

For oil-based (immiscible) lubricants of Jayaprakash, V. et al., 2020, specifically FIG. 3A, the obtained maximum efficiencies range from 42% to 52%. However, this efficiency was only obtained with immiscible lubricant, which limits the applicability of the devices. Notably, where miscible lubricant was used, efficiency was much lower (only 20%), as described previously.

For PCT Application Publication No. WO 2013/114114, specifically FIG. 9, an increase in flow rate for a pressure at syringe of 0.2 bar can be calculated. Without core annular flow, the flow rate of glycerol is approximately 64 μL/min. With core annular flow, the flow rate is 1500 μL/min at Qₒ/Qᵢ=2.0, yielding a 1500/64=23.4 times improvement in flow rate. Under these conditions, with a

$$\frac{{μ}_{i}}{{μ}_{o}} = 400 ,$$

the maximum pressure reduction factor is 128. Therefore, an efficiency of 23.4/128=18.3% is estimated.

For U.S. Pat. No. 9,737,662, and specifically from Table 1, the average core annular flow injection time is 6.14 seconds. Using the conservative estimate of glycerol run of 85 seconds provided in the work, an increase in flow rate of 13.84 times is estimated. A BD 3 mL syringe and a BD 1 mL syringe compressed simultaneously results in a Qₒ/Qᵢ of 0.305. Under these conditions, the maximum pressure reduction factor is 40.45, resulting in efficiency of 13.84/40.45=34%. Notably, this result was only achieved with perfectly vertical operation of the device. Tilting the device would have resulted in lower efficiency. Therefore, this result too is limiting on the applicability of the device.

FIG. 2 panel C shows data collected for fluids with an inner viscosity of 500 cP, an outer viscosity of 1 cP, with a Qₒ/Qᵢ ratio of 0.12, at a rate of 6 mL/min through a 27G needle with inner diameter of 210 μm and a length of 0.0127 m. The force measured is that which is applied to the two plungers of a device, wherein the first plunger has a diameter of 1.2 cm (dₚ₁) and the second plunger has a diameter of 0.467 cm (dₚ₂). The expected injection force for a device without core annular flow is:

$$F = P * A = \frac{128 {μ}_{i} L {Q}_{i}}{π {d}^{4}} * \frac{π}{4} ( {d}_{p 1}^{2} + {d}_{p 2}^{2} ) = 1733 N \qquad ( 21 )$$

This results in a pressure reduction factor of 1733/20=86.66. For the given viscosity ratio and Qₒ/Qᵢ ratio, the ideal pressure reduction factor is 161. Therefore, the device efficiency is:

$$η = \frac{86.66}{161} = 53.8 \% .$$

Devices with the characteristics described herein have reached efficiencies exceeding 70% for a wide range of viscosities and flowrates.

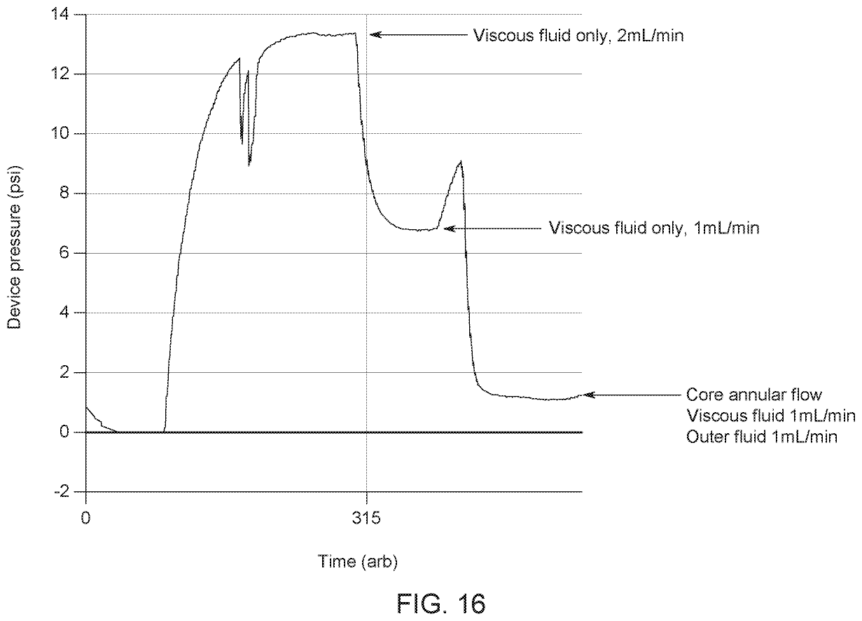

FIG. 16 shows testing of a core annular flow device with a pressure sensor incorporated upstream of the hub and constriction. The viscous inner fluid has a viscosity of 17.8 cP, and the outer fluid has a viscosity of 1 cP. Initially, only the viscous fluid was flowed through the device at a rate of 1 mL/min, yielding a pressure of 6.79 psi. The outer lower-viscosity flow was then initiated at a rate of 1 mL/min, initiating core annular flow and resulting in a pressure of 1.17 psi, for a pressure reduction factor of 5.8. The ideal pressure reduction factor for this viscosity ratio and flow rate ratio is 7.98. This yields a device efficiency of 73%. Device and channel efficiencies up to at least such high values have been achieved using miscible first (inner) fluids and lubricants, immiscible first (inner) fluids and lubricants, and vertical device/channel orientations.

Such measurements were made for a range of viscosity ratios and flow rates and as shown in Table 3.

TABLE 3

| | Viscosity Ratio | Inner fluid flow | Flow rate ratio | |
|---|---|---|---|---|
| Trial # | μᵢ/μₒ | rate (mL/min) | Qo/Qi | Efficiency |
| 1 | 17.8 | 1 | 1 | 73% |
| 2 | 17.8 | 1 | 0.5 | 68% |
| 3 | 17.8 | 1 | 0.2 | 56% |
| 4 | 17.8 | 1 | 0.1 | 45% |
| 5 | 17.8 | 4 | 1 | 69% |
| 6 | 17.8 | 4 | 0.5 | 72% |
| 7 | 17.8 | 4 | 0.2 | 76% |
| 8 | 650 | 1 | 1 | 65% |
| 9 | 650 | 1 | 0.5 | 54% |
| 10 | 650 | 1 | 0.2 | 47% |
| 11 | 650 | 4 | 1 | 63% |
| 12 | 650 | 4 | 0.5 | 54% |
| 13 | 650 | 4 | 0.2 | 57% |

Certain embodiments of the present disclosure were described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

1.-135. (canceled)

136. A fluid delivery device comprising a channel extending from an inlet to an outlet, wherein the channel is sized and shaped such that a first fluid can flow through the channel from the inlet to and through the outlet under a core annular flow.

137. The device of claim 136, wherein the channel is sized and shaped to stabilize against viscous displacement during flow of the fluid through the channel.

138. The device of claim 136, wherein throughput flow through the outlet is greater than total compliance flow of the core annular flow.

139. The device of claim 136, wherein the channel is sized and shaped such that the first fluid does not flow into or through the second inlet.

140. The device of claim 136, wherein the device is shaped to establish core annular flow that has resistance and compliance components designed to stabilize the core annular flow against viscous displacement when the fluid is flowed through the device.

141. The device of claim 136, wherein the device comprises a viscous fluid passageway and a lubricant passageway, wherein the one or more features comprises a choke point within the lubricant passageway.

142. The device of claim 136, comprising a constriction in the channel disposed between the inlet and the outlet, the constriction having an interior shape that comprises a convex portion relative to an interior of the channel.

143. The device of claim 142, wherein the constriction tapers relatively more sharply with position at a larger diameter end of the constriction and relatively less sharply with position at a smaller diameter end.

144. The device of claim 142, wherein the constriction has a length of no more than 10 mm.

145. The device of claim 142, wherein the inlet and the constriction are disposed no more than 5 mm apart.

146. The device of claim 136, wherein (i) the channel comprises a second inlet, (ii) the inlet is an inlet for the first fluid, and (iii) the second inlet is an inlet for a lubricant that separates the first fluid from the interior surface when flowing under the core annular flow and the lubricant is less viscous than the first fluid, wherein a ratio of area of the inlet to area of the second inlet is in a range of from 0.1 to 5.

147. The device of claim 136, wherein no diameter of the channel is more than 2 cm.

148. The device of claim 136, comprising (i) a hub comprising the channel and the outlet and (ii) a needle attached to the hub and a lumen in fluid communication with the channel, wherein the needle comprises a tapered entrance to the lumen, a largest inner diameter of the tapered entrance corresponding to a smallest inner diameter of the outlet of the hub, and a smallest inner diameter of the tapered entrance corresponding to an inner diameter of the lumen.

149. The device of claim 136, comprising (i) a hub comprising the channel and the outlet and (ii) a needle attached to the hub and a lumen in fluid communication with the channel, wherein the needle comprises a proximal blunt-end and comprises a face perpendicular to an axis of fluid flow through the needle and an inner diameter at the face corresponds to a smallest inner diameter of the outlet of the hub.

150. The device of claim 136, wherein the device is shaped to establish core annular flow that matches a resistance of lubricant fluidic pathway(s) to a resistance of viscous fluidic pathway(s) to within 1000% when the fluid is flowed through the device.

151. The device of claim 136, wherein the device is shaped to establish core annular flow that matches a resistance of lubricant fluidic pathway(s) to a resistance of viscous fluidic pathway(s) to within 10000% when the fluid is flowed through the device.

152. The device of claim 136, wherein the device matches an upstream pressure of a lubricant reservoir and a viscous fluid reservoir when the fluid is flowed through the device.

153. The device of claim 136, wherein the first fluid has a viscosity of greater than 400 cP.

154. The device of claim 136, wherein the core annular flow has an efficiency (η) of greater than 20% when the first fluid and lubricant are miscible and the axis of fluid flow through the outlet is within 15 degrees of a line perpendicular to gravity.

155. The device of claim 136, wherein the device comprises one or more fluidic dampers to isolate compliant elements, thereby reducing viscous displacement due to compliance flow, when the fluid is flowed through the device.

156. A device for delivery of a viscous fluid, wherein the device is shaped to establish a core annular flow for delivery of the viscous fluid from a barrel of the device through an outlet when the fluid is flowed through the device, and wherein the device comprises one or more features designed to reduce and/or minimize lubricant compliance (Cₗ).

157. The device of claim 156, wherein the device comprises one or more features designed to reduce and/or minimize lubricant compliance (Cₗ) defined as
$${C}_{l} = \frac{\partial V}{\partial P}$$
(change in volume per change in pressure), wherein Cₗ is no greater than
$$1 {0}^{- 11} [ \frac{{s}^{2} \cdot {m}^{4}}{k g} ] .$$

158. The device of claim 156, wherein the one or more features designed to reduce and/or minimize lubricant compliance comprises a mechanism for elimination of air bubbles in the device.

159. A device for delivery of a viscous fluid, wherein the device is shaped to establish a core annular flow for delivery of the viscous fluid from a barrel of the device through an outlet when the fluid is flowed through the device, and wherein the device comprises one or more features designed to correlate the upstream resistances of the lubricant and viscous fluid (Rₗ and Rᵥ).

160. The device of claim 159, wherein the device comprises a viscous fluid passageway and a lubricant passageway, wherein the one or more features comprises a choke point within the lubricant passageway.

161. The device of claim 159, wherein ΔPupstream, v/ΔPupstream,l is no greater than 10,000.

162. A device for delivery of a viscous fluid, wherein the device is shaped to establish a core annular flow for delivery of the viscous fluid from a barrel of the device through an outlet when the fluid is flowed through the device, and wherein the device comprises one or more features designed to reduce and/or minimize pressure disturbances (∂P/∂t).

163. The device of claim 162, wherein the device comprises a lubricant reservoir and a viscous reservoir, and wherein the one or more features designed to reduce and/or minimize pressure disturbances comprises a common pressure headspace that is shared between the lubricant and viscous reservoirs.

164. The device of claim 162, wherein the one or more features designed to reduce and/or minimize pressure disturbances comprises a passageway or other region with a size sufficiently small to incur viscous losses.

165. The device of claim 162, wherein the device comprises a hub that initiates the core annular flow, wherein the one or more features designed to reduce and/or minimize pressure disturbances comprises a damping element placed between the hub and a compliant upstream element.
